(19) United States
(12) Patent Application Publication
(10) Pub. No.: US 2001/0051855 A1
(43) Pub. Date: Dec. 13, 2001

(54) Computationally targeted evolutionary design

(75) Inventors: Zhen-Gang Wang, Pasadena, CA (US); Christopher A. Voigt, Pasadena, CA (US); Stephen L. Mayo, Pasadena, CA (US); Frances H. Arnold, Pasadena, CA (US)

Correspondence Address: DARBY & DARBY P.C., 805 Third Avenue, New York, NY, 10022, US

(73) Assignee: California Institute of Technology

(21) Appl. No.: 09/795,500

(22) Filed: Feb. 16, 2001

Related U.S. Application Data
(60) Provisional application No. 60/183,171, filed on Feb. 17, 2000.

(51) Int. Cl. G06F 19/00; G01N 33/48; G01N 33/50

(52) U.S. Cl. 702/19; 702/20

(57) ABSTRACT

The invention relates to improved methods for directed evolution of polymers, including directed evolution of nucleic acids and proteins. Specifically, the methods of the invention include analytical methods for identifying “structurally tolerant” residues of a polymer. Mutations of these, structurally tolerant residues are less likely to adversely affect desirable properties of a polymer sequence. The invention further provides improved methods for directed evolution wherein the structurally tolerant residues of a polymer are selectively mutated. Computer systems for implementing analytical methods of the invention are also provided.

This application claims priority under 35 U.S.C. § 119(e) to co-pending U.S. Provisional Patent Application Ser. No. 60/183,171 filed on Feb. 17, 2000, which is incorporated herein by reference in its entirety.

Numerous references, including patents, patent applications and various publications, are cited and discussed in this specification. The citation and/or discussion of such references is provided merely to clarify the description of the invention and is not an admission that any such reference is “prior art” to the invention described herein. All references cited and discussed in this specification are incorporated herein by reference in their entirety and to the same extent as if each reference was individually incorporated by reference.

### 1. FIELD OF THE INVENTION

The invention relates to biomolecular engineering and design, including methods for the engineering and design of biopolymers such as proteins and nucleic acids. In particular, the invention relates to methods for directed evolution, including in vitro directed evolution, of biopolymers such as proteins and nucleic acids. The invention also relates to computational methods for identifying residues of a biopolymer (e.g., nucleotide residues of a nucleic acid or amino acid residues of a polypeptide) where mutations may produce beneficial results, such as one or more improved properties. Preferably, improvements are obtained while minimally disrupting a desired biopolymer property, such as stability or functionality. Disruption is less likely when biopolymers are mutated at structurally tolerant mutation sites determined according to the invention. This provides a targeted approach for obtaining mutant or hybrid biopolymers with improved properties using directed evolution techniques. More particularly, the invention is useful in the design of hybrid polypeptides having new or improved properties.

### 2. BACKGROUND OF THE INVENTION

The invention is concerned primarily with biopolymers such as polynucleotides (chains of nucleic acids) and polypeptides (proteins). Proteins are polypeptides that are useful to living organisms. For example, they provide structures in the body, do physical or chemical work, or act as catalysts for chemical reactions (i.e. as enzymes). Proteins are made by cells according to genetic information encoded, translated and transcribed by polynucleotides (DNA and RNA). It is often desirable to modify proteins so that they have new or improved properties. For example, a protein may be altered to increase its biological activity (e.g. its potency as an enzyme), or to improve its stability under different environmental conditions (e.g. temperature, organic solvent), or to change its function (e.g. to catalyze a different chemical reaction).

Nature makes these kinds of alterations in many ways, including for example genetic mutations, or changes due to the recombination of genetic material such as occurs from sexual reproduction. Changes that are beneficial tend to be preserved from generation to generation, while truly harmful changes may disappear over time, in a process called evolution. Changes which are neutral, i.e. neither helpful nor harmful, may also be preserved by default. This is a very long process, and tends to produce random changes which are then tested for survival by the environment. Scientists looking for proteins with improved properties have had the very difficult task of searching for changes in proteins at random, from the vast numbers of potential natural sources that are available. Changes that are desirable may not be produced or preserved by nature. Breeding experiments can be done to provide additional sources for genetic variation, tending toward traits of interest, but these techniques also are exceedingly slow, costly, and resource intensive. They are very inefficient, and may not produce desired results. For example, proteins that act as enzymes to break down other proteins can be used as stain-removing ingredients of a laundry detergent, but these proteins may have to work at higher temperatures than in nature. Identifying proteins with desirable characteristics from nature, such as enzymes with improved heat resistance (thermostability) has been a haphazard and difficult process. Accordingly, there has been a need for new ways to modify proteins, or the polynucleotides which encode them, to produce new proteins with improved properties.

Two separate techniques commonly used to alter the properties of proteins and other biological molecules are directed evolution and computational design.

Directed Evolution

Directed evolution techniques attempt to alter the properties of a biopolymer (e.g., a protein or a nucleic acid) by accumulating stepwise improvements through interations of random mutagenesis, recombination and screening (see, e.g., Moore & Arnold, Nature Biotechnology 1996, 14:458; Miyazaki et al., J. Mol. Biol. 2000, 297:1015-1026; Arnold, Adv. Protein Chem. 2000, 55: ix-xi). Broadly speaking, these methods work by speeding up the natural processes of evolution. Changes in genetic material (e.g. mutations) are rapidly and artificially induced, typically in cells that can be easily and quickly grown in cell culture (e.g. outside the body). The resulting mutants are rapidly evaluated to identify new or improved properties or changes of interest. Genetic recombination methods have been widely applied to accelerate in vitro protein evolution (See, e.g., Stemmer, Proc. Natl. Acad. Sci. 1994, 91 :10747; Stemmer, Nature 1994, 370:389; Zhao & Arnold, Nucleic Acids Res.1997, 25:1307; Zhao et al., Nature Biotechnology 1998, 49:290). Examples of in vitro recombination methods include DNA shuffling, random-priming recombination, and the staggered extension process (StEP) (see, Arnold & Wintrode, Enzymes, Directed Evolution, in Encyclopedia of bioprocess technology: fermentation, biocatalysis, and bioseparation 1999,2:971).

Computational Design

Computational design, by contrast, has developed separately from directed evolution and is a fundamentally different approach (Street & Mayo, Structure 1999 7: R105). Unlike the essentially random approach of directed evolution, computational design attempts to predict and then make the changes or mutations that will be beneficial or useful. Thus, the general objective of computational design is to identify particular interactions in a protein (or other biopolymer) that lead to desirable properties, and then modify the biopolymer sequence to optimize those interactions. For example, a force field model is typically used to quantitatively describe interactions between amino acid residues in a protein. An amino acid sequence may then be computed, at least in theory, to globablly optimize these interactions (see, e.g., Malakaukas & Mayo, Nature Structural Biology 1998,5:470; Dahiyat & Mayo, Science 1997, 278:82).

The Sequence Space

Computational design can effectively search a large sequence space, that is, a large number of sequences (e.g., >10²⁶). See, Dahiyat & Mayo, Science 1997278:82). However, the technique is currently limited by the size of the biopolymer. The largest full sequence design accomplished to date is a 28-mer zinc finger protein (id.). Partial designs (e.g. limiting the number of residues calculated) can be done to improve the stability of proteins up to about 70 amino acids. Moreover, the technique currently is based on calculating the molecule's conformational energy, i.e. the relative energy of the molecule's folded and unfolded states. Thus, current computational methods have only been used to improve a molecule's stability. The technique has not been used to improve other properties of biopolymers, such as activity, selectivity, efficiency, or other characteristics of biological fitness.

Directed evolution methods, by contrast, have the benefit of improving any property in a molecule that can be detected and/or captured by a screen, for example catalytic activity of an enzyme. For example, one effective and widely used directed evolution method involves production of a library of mutants from a parent sequence, e.g., by using error-prone PCR to produce random point mutations (see, Moore & Arnold, Nature Biotechnology 1996, 14:458; Miyazaki et al, J. Mol. Biol. 2000, 297:1015-1026). However, the technique is limited by several factors, one of which is the practical size of the screen (Zhao & Arnold, Protein Engineering 1999, 12:47). Increasing the number of mutants screened enables the user to sample a larger fraction of possible sequences (i.e., the “sequence space”) and therefore provides better improvements in the properties of interest. However, the most mutants that may be observed in any real screen or selection is between about 10³ to 10¹², depending upon the specific method. In comparison, however, an average protein of 300 residues will have at least 10³⁹⁰ possible amino acid combinations. Thus, any real screening or selection assay can only search a very small fraction of the possible sequences.

Moreover, the probability that any single random mutation will improve a property of the parent sequence is small, and the probability of improvement decreases rapidly when multiple simultaneous mutations are made. Furthermore, the negligible probability that two or three mutations occur in a single codon and the significant biases of error-prone PCR severely restrict the possible amino acid substitutions which may be searched. These effects can be at least partially overcome by intensely mutagenizing a limited number of positions in the parent sequence (see, e.g., Skandalis et al., Chem. Biol. 1997, 4:889; and Miyazaki & Arnold, J. Molecular Evolution 1999, 49:716). However, to successfully implement such a technique, it is necessary to first identify the particular residues where selective mutagenesis is likely to be beneficial, as beneficial mutations often appear far from sites, such as catalytic sites, that would be predicted heuristically (Moore & Arnold, Nature Biotechnology 1996, 14:458; Miyazaki et al., J. Mol. Biol. 2000, 297:1015-1026).

Exemplary Directed Evolution Methods

Exmplary directed evolution techniques include DNA shuffling, random-primer extension, and StEP recombination.

In DNA shuffling, the parental DNA is enzymatically digested into fragments which can be reassembled into offspring genes (Stemmer, Proc. Natl. Acad. Sci. 1994 91:10747; Stemmer, Nature 1994, 370:389; Zhao & Arnold, Nucleic Acids Res.1997, 25:1307).

In the random-primer method, template DNA sequences are primed with random-sequence primers and then extended by DNA polymerase to create fragments. The template is removed and the fragments are reassembled into full length genes, as in the final step of DNA shuffling (Shao et al., Nuc. Acids Res. 1998, 26:681). In each of these methods, the number of cut points can be increased by starting with smaller fragments or by limiting the extension reaction.

StEP recombination differs from the first two methods because it does not use gene fragments (Zhao et al., Nat. Biotechnology 1998, 49:290). The template genes are primed and extended before denaturation and reannealing. As the fragments grow, they reanneal to new templates and thus combine information from multiple parents. This process is cycled hundreds of times until a full length offspring gene is formed.

Thus, there is presently a need in the art for improved methods of designing biopolymers such as proteins and nucleic acids. Moreover, there exists a need for better methods for improving one or more properties of a biopolymer. There further exists a need for improved methods of directed evolution that overcome, at least partially, any one or more of the above-described problems in the art. For example, there is a need in the art to identify residues of a molecule (e.g., a biopolymer such as a protein or nucleic acid) where mutagenesis is likely to be beneficial and/or improve one or more properties.

### 3. SUMMARY OF THE INVENTION

Mutations to a polymer (e.g., a polypeptide or nucleic acid) are less likely to have an adverse affect on the “fitness” of the polymer when only “structurally tolerant” residues are mutated. In particular, the structurally tolerant residues are preferably ones that have few and/or weak (if any) coupling interactions with other residues in the polymer. Applicants have discovered novel techniques for identifying structurally tolerant residues in a polymer sequence. These methods are straightforward and are computationally tractable. Accordingly, a skilled artisan can readily use these methods to identify the residues of a particular polymer sequence that are structurally tolerant, and may selectively mutate those residues to generate compatible mutants that do not adversely affect that particular polymer's properties of interest. Applicants have discovered that such mutants are more likely to have one or more properties of interest that are improved over the properties of the parent polymer. There is significant overlap between tolerant mutations and beneficial mutations. Thus, by selectively mutating structurally tolerant residues a skilled artisan may more readily and efficiently identify novel sequences with improved properties than if the artisan randomly mutated the polymer.

The invention therefore provides methods for selecting residues of a polymer sequence for mutation by obtaining or determining the structural tolerance for residues of the polymer sequence, and selecting structurally tolerant residues for mutation. The polymers may be any type of polymer, including biopolymers such as, but not limited to, nucleic acids (comprising a sequence of nucleotide residues) and proteins or polypeptides (comprising a sequence of amino acid residues). The invention also provides numerous methods for determining the structural tolerance of residues in a polymer including, in preferred embodiments, the site entropy of the residues.

The invention also provides methods for directed evolution of polymers. In such methods, a parent sequence may be provided that has one or more properties of interest and one or more structurally tolerant residues selected for mutation. One or more mutant polymers may then be generated from the parent polymer sequence in which one or more of the selected structurally tolerant residues are mutated, and these mutants are then preferably screened for the one or more properties of interest. Mutants are then selected where one or more of the properties of interest is modified and, preferably, is improved. In preferred embodiments, the directed evolution methods of the invention are iteratively repeated, and selected mutants are used as parent polymer sequences in subsequent iterations of the method.

The invention can also be used to identify parent molecules or families of parent molecules (e.g. preferred parent genes or gene families) for mutation. For example, a particular biochemical reaction may be facilitated by more than one enzyme or enzyme family, encoded by more than one gene or gene family. These genes or gene families can be evaluated to determine which are more likely, when altered (e.g. by directed evolution), to produce desirable improvements.

Computer systems are also provided that may be used to implement the analytical methods of the invention, including methods of identifying structurally tolerant residues in a polymer sequence and/or selecting such residues for mutation (e.g., as part of a directed evolution method). These computer systems comprise a processor interconnected with a memory that contains one or more software components. In particular, the one or more software components include programs that cause the processor to implement steps of the analytical methods described herein. The software components may comprise additional programs and/or files including, for example, sequence or structural databases of polymers.

Computer program products are further provided, which comprise a computer readable medium, such as one or more floppy disks, compact discs (e.g., CD-ROMS or RW-CDS), DVDs, data tapes, etc., that have one or more software components encoded thereon in computer readable form. In particular, the software components may be loaded into the memory of a computer system and may then cause a processor of the computer system to execute steps of the analytical methods described herein. The software components may include additional programs and/or files including databases, e.g., of polymer sequences and/or structures.

### 4. BRIEF DESCRIPTION OF THE DRAWINGS

FIG. 1 is a flow diagram illustrating an exemplary embodiment of the methods of the invention.

FIG. 2 shows an exemplary computer system that may be used to implement analytical methods of the invention.

FIG. 3 is a plot of the probability distribution P(c) that a positive mutation (i.e., a mutation increasing the protein fitness, F) occurs at a residue having c coupled interactions. The probability distribution is shown for two different fitness values: F=0.0 (∘); and F 17.0 (▴).

FIGS. 4A-B are plots showing the site entropy profile sᵢ (FIG. 4A) and %-solvent exposure (FIG. 4B) for amino acid residues 5-268 of subtilisin E protein.

FIGS. 5A-B show the probability distribution P(sᵢ) of site entropy values sᵢ in subtilisin E protein (FIG. 5A) and T4 lysozyme protein (FIG. 5B).

FIG. 6 shows the three dimension crystal structure of subtilisin E protein, with the site entropy sᵢ of each amino acid residue indicated by its color: yellow, 2.16<sᵢ<3.00; red, 1.31<sᵢ<2.16; gray, sᵢ<1.31.

FIG. 7 shows the off-rate k_{off} of the 4-4-20 antibody mutants plotted against the entropy of the sites where the beneficial mutations occurred.

FIG. 8 is a representative plot of percent functional improvement versus entropy for T4 lysozyme in a model according to the invention.

FIG. 9 shows a representative comparison of T4 lysozyme entropy calculated according to mean-field algorithm and dead-end elimination algorithms.

### 5. DETAILED DESCRIPTION OF THE INVENTION

The invention overcomes problems in the prior art and provides novel methods which can be used for directed evolution of biopolymers such as proteins and nucleic acids. In particular, the invention provides methods which can be used to identify residues of a polymer where mutations are most likely to produce one or more improved properties. By preferentially mutating these residues, the sequence space for a given polymer may be more efficiently searched. Mutant or variant polymers having one or more improved properties may be more readily identified while simultaneously reducing the number(s) of mutants screened.

The inventors have discovered, in particular, that the probability of a beneficial mutation occurring at a highly coupled residue decreases significantly as the “fitness” of the parent polymer increases. Highly coupled residues in a polymer will generally require several simultaneous mutations at other residues to demonstrate improvement, e.g., in a directed evolution experiment. As the polymer in the directed experiment becomes more highly optimized (i.e. becomes more “fit”), the probability that this occurs decreases rapidly due to the limited mutation rate and library size. However, fewer simultaneous mutations are generally required for mutations at uncoupled or weakly coupled residues to improve a particular property of the polymer. Thus, it is more likely that a beneficial mutation will occur at these uncoupled or weakly coupled mutations in a directed evolution experiment.

The details of the invention are described below by means of numerous examples of increasing detail and specificity. These examples are provided only to illustrate preferred embodiments of the invention. However, the invention is not limited to the particular embodiments, and many modifications and variations of the invention will be apparent to those skilled in the art. Such modifications and variations are therefore also considered part of the invention.

5.1. Definitions

The terms used in this specification generally have their ordinary meanings in the art, within the context of this invention and in the specific context where each term is used. Certain terms are discussed below or elsewhere in the specification, to provide additional guidance to the practitioner in describing the compositions and methods of the invention and how to make and use them. The scope an meaning of any use of a term will be apparent from the specific context in which the term is used.

Molecular Biology

The term “molecule” means any distinct or distinguishable structural unit of matter comprising one or more atoms, and includes, for example, polypeptides and polynucleotides.

The term “polymer” means any substance or compound that is composed of two or more building blocks (‘mers’) that are repetitively linked together. For example, a “dimer” is a compound in which two building blocks have been joined together; a “trimer” is a compound in which three building blocks have been joined together; etc. The individual building blocks of a polymer are also referred to herein as “residues”.

A “biopolymer” is any polymer having an organic or biochemical utility or that is produced by a cell. Preferred biopolymers include, but are not limited to, polynucleotides, polypeptides and polysaccharides.

The term “polynucleotide” or “nucleic acid molecule” refers to a polymeric molecule having a backbone that supports bases capable of hydrogen bonding to typical polynucleotides, wherein the polymer backbone presents the bases in a manner to permit such hydrogen bonding in a specific fashion between the polymeric molecule and a typical polynucleotide (e.g., single-stranded DNA). Such bases are typically inosine, adenosine, guanosine, cytosine, uracil and thymidine. Polymeric molecules include “double stranded” and “single stranded” DNA and RNA, as well as backbone modifications thereof (for example, methylphosphonate linkages).

Thus, a “polynucleotide” or “nucleic acid” sequence is a series of nucleotide bases (also called “nucleotides”), generally in DNA and RNA, and means any chain of two or more nucleotides. A nucleotide sequence frequently carries genetic information, including the information used by cellular machinery to make proteins and enzymes. The terms include genomic DNA, cDNA, RNA, any synthetic and genetically manipulated polynucleotide, and both sense and antisense polynucleotides. This includes single- and double-stranded molecules; i.e., DNA-DNA, DNA-RNA, and RNA-RNA hybrids as well as “protein nucleic acids” (PNA) formed by conjugating bases to an amino acid backbone. This also includes nucleic acids containing modified bases, for example, thio-uracil, thio-guanine and fluoro-uracil.

The polynucleotides herein may be flanked by natural regulatory sequences, or may be associated with heterologous sequences, including promoters, enhancers, response elements, signal sequences, polyadenylation sequences, introns, 5′- and 3′-non-coding regions and the like. The nucleic acids may also be modified by many means known in the art. Non-limiting examples of such modifications include methylation, “caps”, substitution of one or more of the naturally occurring nucleotides with an analog, and internucleotide modifications such as, for example, those with uncharged linkages (e.g., methyl phosphonates, phosphotriesters, phosphoroamidates, carbamates, etc.) and with charged linkages (e.g., phosphorothioates, phosphorodithioates, etc.). Polynucleotides may contain one or more additional covalently linked moieties, such as proteins (e.g., nucleases, toxins, antibodies, signal peptides, poly-L-lysine, etc.), intercalators (e.g., acridine, psoralen, etc.), chelators (e.g., metals, radioactive metals, iron, oxidative metals, etc.) and alkylators to name a few. The polynucleotides may be derivatized by formation of a methyl or ethyl phosphotriester or an alkyl phosphoramidite linkage. Furthermore, the polynucleotides herein may also be modified with a label capable of providing a detectable signal, either directly or indirectly. Exemplary labels include radioisotopes, fluorescent molecules, biotin and the like. Other non-limiting examples of modification which may be made are provided, below, in the description of the invention.

The term “oligonucleotide” refers to a nucleic acid, generally of at least 10, preferably at least 15, and more preferably at least 20 nucleotides, preferably no more than 100 nucleotides, that is hybridizable to a genomic DNA molecule, a cDNA molecule, or an mRNA molecule encoding a gene, mRNA, cDNA, or other nucleic acid of interest. Oligonucleotides can be labeled, e.g., with ³²P-nucleotides or nucleotides to which a label, such as biotin or a fluorescent dye (for example, Cy3 or Cy5) has been covalently conjugated. Generally, oligonucleotides are prepared synthetically, preferably on a nucleic acid synthesizer. Accordingly, oligonucleotides can be prepared with non-naturally occurring phosphoester analog bonds, such as thioester bonds, etc.

A “polypeptide” is a chain of chemical building blocks called amino acids that are linked together by chemical bonds called “peptide bonds”. The term “protein” refers to polypeptides that contain the amino acid residues encoded by a gene or by a nucleic acid molecule (e.g., an mRNA or a cDNA) transcribed from that gene either directly or indirectly. Optionally, a protein may lack certain amino acid residues that are encoded by a gene or by an mRNA. For example, a gene or mRNA molecule may encode a sequence of amino acid residues on the N-terminus of a protein (i.e., a signal sequence) that is cleaved from, and therefore may not be part of, the final protein. A protein or polypeptide, including an enzyme, may be a “native” or “wild-type”, meaning that it occurs in nature; or it may be a “mutant”, “variant” or “modified”, meaning that it has been made, altered, derived, or is in some way different or changed from a native protein or from another mutant.

“Amplification” of a polynucleotide denotes the use of polymerase chain reaction (PCR) to increase the concentration of a particular DNA sequence within a mixture of DNA sequences. For a description of PCR see Saiki et al., Science 1988, 239:487.

A “ligand” is, broadly speaking, any molecule that binds to another molecule. In preferred embodiments, the ligand is either a soluble molecule or the smaller of the two molecule or both. The other molecule is referred to as a “receptor”. In preferred embodiments, both a ligand and its receptor are molecules (preferably proteins or polypeptides) produced by cells. Preferably, a ligand is a soluble molecule and the receptor is an integral membrane protein (i.e., a protein expressed on the surface of a cell). The binding of a ligand to its receptor is frequently a step of signal transduction within a cell. Other exemplary ligand-receptor interactions include, but are not limited to, binding of a hormone to a hormone receptor (for example, the binding of estrogen to the estrogen receptor) and the binding of a neurotransmitter to a receptor on the surface of a neuron.

A “gene” is a sequence of nucleotides which code for a functional “gene product”. Generally, a gene product is a functional protein. However, a gene product can also be another type of molecule in a cell, such as an RNA (e.g., a tRNA or a rRNA). For the purposes of the invention, a gene product also refers to an mRNA sequence which may be found in a cell. For example, measuring gene expression levels according to the invention may correspond to measuring mRNA levels. A gene may also comprise regulatory (i.e., non-coding) sequences as well as coding sequences Exemplary regulatory sequences include promoter sequences, which determine, for example, the conditions under which the gene is expressed. The transcribed region of the gene may also include untranslated regions including introns, a 5′-untranslated region (5′-UTR) and a 3′-untranslated region (3′-UTR).

A “coding sequence” or a sequence “encoding” an expression product, such as a RNA, polypeptide, protein or enzyme, is a nucleotide sequence that, when expressed, results in the production of that RNA, polypeptide, protein or enzyme; i.e., the nucleotide sequence “encodes” that RNA or it encodes the amino acid sequence for that polypeptide, protein or enzyme.

A “promoter sequence” is a DNA regulatory region capable of binding RNA polymerase in a cell and initiating transcription of a downstream (3′ direction) coding sequence. A promoter sequence is typically bounded at its 3′ terminus by the transcription initiation site and extends upstream (5′ direction) to include the minimum number of bases or elements necessary to initiate transcription at levels detectable above background. Within the promoter sequence will be found a transcription initiation site (conveniently found, for example, by mapping with nuclease S1), as well as protein binding domains (consensus sequences) responsible for the binding of RNA polymerase.

A coding sequence is “under the control of” or is “operatively associated with” transcriptional and translational control sequences in a cell when RNA polymerase transcribes the coding sequence into RNA, which is then trans-RNA spliced (if it contains introns) and, if the sequence encodes a protein, is translated into that protein.

The term “express” and “expression” means allowing or causing the information in a gene or DNA sequence to become manifest, for example producing RNA (such as rRNA or mRNA) or a protein by activating the cellular functions involved in transcription and translation of a corresponding gene or DNA sequence. A DNA sequence is expressed by a cell to form an “expression product” such as an RNA (e.g., a mRNA or a rRNA) or a protein. The expression product itself, e.g., the resulting RNA or protein, may also said to be “expressed” by the cell.

The term “transfection” means the introduction of a foreign nucleic acid into a cell. The term “transformation” means the introduction of a “foreign” (i.e., extrinsic or extracellular) gene, DNA or RNA sequence into a host cell so that the host cell will express the introduced gene or sequence to produce a desired substance, in this invention typically an RNA coded by the introduced gene or sequence, but also a protein or an enzyme coded by the introduced gene or sequence. The introduced gene or sequence may also be called a “cloned” or “foreign” gene or sequence, may include regulatory or control sequences (e.g., start, stop, promoter, signal, secretion or other sequences used by a cell's genetic machinery). The gene or sequence may include nonfunctional sequences or sequences with no known function. A host cell that receives and expresses introduced DNA or RNA has been “transformed” and is a “transformant” or a “clone”. The DNA or RNA introduced to a host cell can come from any source, including cells of the same genus or species as the host cell or cells of a different genus or species.

The terms “vector”, “cloning vector” and “expression vector” mean the vehicle by which a DNA or RNA sequence (e.g., a foreign gene) can be introduced into a host cell so as to transform the host and promote expression (e.g., transcription and translation) of the introduced sequence. Vectors may include plasmids, phages, viruses, etc. and are discussed in greater detail below.

A “cassette” refers to a DNA coding sequence or segment of DNA that codes for an expression product that can be inserted into a vector at defined restriction sites. The cassette restriction sites are designed to ensure insertion of the cassette in the proper reading frame. Generally, foreign DNA is inserted at one or more restriction sites of the vector DNA, and then is carried by the vector into a host cell along with the transmissible vector DNA. A segment or sequence of DNA having inserted or added DNA, such as an expression vector, can also be called a “DNA construct.” A common type of vector is a “plasmid”, which generally is a self-contained molecule of double-stranded DNA, usually of bacterial origin, that can readily accept additional (foreign) DNA and which can readily introduced into a suitable host cell. A large number of vectors, including plasmid and fungal vectors, have been described for replication and/or expression in a variety of eukaryotic and prokaryotic hosts.

The term “host cell” means any cell of any organism that is selected, modified, transformed, grown or used or manipulated in any way for the production of a substance by the cell. For example, a host cell may be one that is manipulated to express a particular gene, a DNA or RNA sequence, a protein or an enzyme. Host cells may be cultured in vitro or one or more cells in a non-human animal (e.g., a transgenic animal or a transiently transfected animal).

The term “expression system” means a host cell and compatible vector under suitable conditions, e.g. for the expression of a protein coded for by foreign DNA carried by the vector and introduced to the host cell. Common expression systems include E. coli host cells and plasmid vectors, insect host cells such as Sf9, Hi5 or S2 cells and Baculovirus vectors, Drosophila cells (Schneider cells) and expression systems, and mammalian host cells and vectors.

The terms “mutant” and “mutation” mean any change in a particular polymer sequence (referred to herein as a “parent sequence”). Thus, in the invention mutations may include, but are not limited to, changes in the nucleotide sequence of a nucleic acid (including changes in the sequence of a gene), and also changes in the amino acid sequence of a protein or polypeptide. In preferred embodiments of the invention, mutations are limited to substitutions of one or more polymer residues (e.g., nucleotide and/or amino acid substitutions). However, mutations of the invention may also include deletions or insertions of one or more residues, such as amino acid and/or nucleotide substitutions or deletions.

The methods of the invention may include steps of comparing parent sequences to each other or a parent sequence to one or more mutants. Such comparisons typically comprise alignments of polymer sequences, e.g., using sequence alignment programs and/or algorithms that are well known in the art (for example, BLAST, FASTA and MEGALIGN, to name a few). The skilled artisan can readily appreciate that, in such alignments, where a mutation contains a residue insertion or deletion, the sequence alignment will introduce a “gap” (typically represented by a dash, “-”, or “Δ”) in the polymer sequence not containing the inserted or deleted residue. Thus, for example, in an embodiment where a mutation introduces a single amino acid deletion in a parent sequence at amino acid residue i, an alignment of the parent and mutant polypeptide sequences will introduce a gap in the mutant sequence that aligns with amino acid residue i of the parent. In such embodiments, therefore, amino acid residue i in the mutant sequence is preferably said to be a “gap” or “deletion”.

The term “heterologous” refers to a combination of elements not naturally occurring. For example, chimeric RNA molecules may comprise an rRNA sequence and a heterologous RNA sequence which is not part of the rRNA sequence. In this context, the heterologous RNA sequence refers to an RNA sequence that is not naturally located within the ribosomal RNA sequence. Alternatively, the heterologous RNA sequence may be naturally located within the ribosomal RNA sequence, but is found at a location in the rRNA sequence where it does not naturally occur. As another example, heterologous DNA refers to DNA that is not naturally located in the cell, or in a chromosomal site of the cell. Preferably, heterologous DNA includes a gene foreign to the cell. A heterologous expression regulatory element is a regulatory element operatively associated with a different gene than the one it is operatively associated with in nature.

The term “homologous”, in all its grammatical forms and spelling variations, refers to the relationship between two proteins that possess a “common evolutionary origin”, including proteins from superfamilies (e.g., the immunoglobulin superfamily) in the same species of organism, as well as homologous proteins from different species of organism (for example, myosin light chain polypeptide, etc.; see, Reeck et al., Cell 1987, 50:667). Such proteins (and their encoding nucleic acids) have sequence homology, as reflected by their sequence similarity, whether in terms of percent identity or by the presence of specific residues or motifs and conserved positions.

The term “sequence similarity”, in all its grammatical forms, refers to the degree of identity or correspondence between nucleic acid or amino acid sequences that may or may not share a common evolutionary origin (see, Reeck et al., supra). However, in common usage and in the instant application, the term “homologous”, when modified with an adverb such as “highly”, may refer to sequence similarity and may or may not relate to a common evolutionary origin.

The term “recombination” and variant spellings thereof, encompasses both “homologous” and “non-homologous” recombination. In its most basic form, recombination is the exchange of biopolymer fragments between two biopolymer sequences. As defined in this invention, sequences may be recombined at the amino acid or nucleic acid level.

The term “homologous recombination” refers to the exchange of biopolymer fragments between two bioploymer sequences at locations where the sequences exhibit regions of sequence homology. In more general biological terms, homologous recombination refers to the insertion of a modified or foreign DNA sequence contained by a first vector into another DNA sequence contained in second vector, or a chromosome of a cell. The first vector targets a specific chromosomal site for homologous recombination. For specific homologous recombination, the first vector will contain sufficiently long region of homology to sequences of the second vector or chromosome to allow complementary binding and incorporation of DNA from the first vector into the DNA of the second vector, or the chromosome.

The term “non-homologous recombination” refers to the exchange of biopolymer fragments between two biopolymer sequences at location where the sequences are not homologous.

A nucleic acid molecule is “hybridizable” to another nucleic acid molecule, such as a cDNA, genomic DNA, or RNA, when a single stranded form of the nucleic acid molecule can anneal to the other nucleic acid molecule under the appropriate conditions of temperature and solution ionic strength (see Sambrook et al., supra). The conditions of temperature and ionic strength determine the “stringency” of the hybridization. For preliminary screening for homologous nucleic acids, low stringency hybridization conditions, corresponding to a Tₘ (melting temperature) of 55° C., can be used, e.g., 5×SSC, 0.1% SDS, 0.25% milk, and no formamide; or 30% formamide, 5×SSC, 0.5% SDS). Moderate stringency hybridization conditions correspond to a higher Tₘ, e.g., 40% formamide, with 5× or 6×SSC. High stringency hybridization conditions correspond to the highest Tₘ, e.g., 50% formamide, 5× or 6×SSC. SSC is a 0.15M NaCl, 0.015M Na-citrate. Hybridization requires that the two nucleic acids contain complementary sequences, although depending on the stringency of the hybridization, mismatches between bases are possible. The appropriate stringency for hybridizing nucleic acids depends on the length of the nucleic acids and the degree of complementation, variables well known in the art. The greater the degree of similarity or homology between two nucleotide sequences, the greater the value of Tₘ for hybrids of nucleic acids having those sequences. The relative stability (corresponding to higher Tₘ) of nucleic acid hybridizations decreases in the following order: RNA:RNA, DNA:RNA, DNA:DNA. For hybrids of greater than 100 nucleotides in length, equations for calculating Tₘ have been derived (see Sambrook et al., supra, 9.50-9.51). For hybridization with shorter nucleic acids, i.e., oligonucleotides, the position of mismatches becomes more important, and the length of the oligonucleotide determines its specificity (see Sambrook et al., supra, 11.7-11.8). A minimum length for a hybridizable nucleic acid is at least about 10 nucleotides; preferably at least about 15 nucleotides; and more preferably the length is at least about 20 nucleotides.

Unless specified, the term “standard hybridization conditions” refers to a Tₘ of about 55° C., and utilizes conditions as set forth above. In a preferred embodiment, the Tₘ is 60° C.; in a more preferred embodiment, the Tₘ is 65° C. In a specific embodiment, “high stringency” refers to hybridization and/or washing conditions at 68° C. in 0.2×SSC, at 42° C. in 50% formamide, 4×SSC, or under conditions that afford levels of hybridization equivalent to those observed under either of these two conditions.

Suitable hybridization conditions for oligonucleotides (e.g., for oligonucleotide probes or primers) are typically somewhat different than for full-length nucleic acids (e.g., full-length cDNA), because of the oligonucleotides' lower melting temperature. Because the melting temperature of oligonucleotides will depend on the length of the oligonucleotide sequences involved, suitable hybridization temperatures will vary depending upon the oligoncucleotide molecules used. Exemplary temperatures may be 37° C. (for 14-base oligonucleotides), 48° C. (for 17-base oligoncucleotides), 55° C. (for 20-base oligonucleotides) and 60° C. (for 23-base oligonucleotides). Exemplary suitable hybridization conditions for oligonucleotides include washing in 6×SSC/0.05% sodium pyrophosphate, or other conditions that afford equivalent levels of hybridization.

The term “isolated” means that the referenced material is removed from the environment in which it is normally found. Thus, an isolated biological material can be free of cellular components, i.e., components of the cells in which the material is found or produced. In the case of nucleic acid molecules, an isolated nucleic acid includes a PCR product, an isolated mRNA, a cDNA, or a restriction fragment. In another embodiment, an isolated nucleic acid is preferably excised from the chromosome in which it may be found, and more preferably is no longer joined to non-regulatory, non-coding regions, or to other genes, located upstream or downstream of the gene contained by the isolated nucleic acid molecule when found in the chromosome. In yet another embodiment, the isolated nucleic acid lacks one or more introns. Isolated nucleic acid molecules include sequences inserted into plasmids, cosmids, artificial chromosomes, and the like. Thus, in a specific embodiment, a recombinant nucleic acid is an isolated nucleic acid. An isolated protein may be associated with other proteins or nucleic acids, or both, with which it associates in the cell, or with cellular membranes if it is a membrane-associated protein. An isolated organelle, cell, or tissue is removed from the anatomical site in which it is found in an organism. An isolated material may be, but need not be, purified.

The term “purified” refers to material that has been isolated under conditions that reduce or eliminate the presence of unrelated materials, i.e., contaminants, including native materials from which the material is obtained. For example, a purified protein is preferably substantially free of other proteins or nucleic acids with which it is associated in a cell; a purified nucleic acid molecule is preferably substantially free of proteins or other unrelated nucleic acid molecules with which it can be found within a cell. The term “substantially free” is used operationally, in the context of analytical testing of the material. Preferably, purified material substantially free of contaminants is at least 50% pure; more preferably, at least 90% pure, and more preferably still at least 99% pure. Purity can be evaluated by chromatography, gel electrophoresis, immunoassay, composition analysis, biological assay, and other methods known in the art.

Methods for purification are well-known in the art. For example, nucleic acids can be purified by precipitation, chromatography (including preparative solid phase chromatography, oligonucleotide hybridization, and triple helix chromatography), ultracentrifugation, and other means. Polypeptides and proteins can be purified by various methods including, without limitation, preparative disc-gel electrophoresis, isoelectric focusing, HPLC, reversed-phase HPLC, gel filtration, ion exchange and partition chromatography, precipitation and salting-out chromatography, extraction, and countercurrent distribution. For some purposes, it is preferable to produce the polypeptide in a recombinant system in which the protein contains an additional sequence tag that facilitates purification, such as, but not limited to, a polyhistidine sequence, or a sequence that specifically binds to an antibody, such as FLAG and GST. The polypeptide can then be purified from a crude lysate of the host cell by chromatography on an appropriate solid-phase matrix. Alternatively, antibodies produced against the protein or against peptides derived therefrom can be used as purification reagents. Cells can be purified by various techniques, including centrifugation, matrix separation (e.g., nylon wool separation), panning and other immunoselection techniques, depletion (e.g., complement depletion of contaminating cells), and cell sorting (e.g., fluorescence activated cell sorting or “FACS”). Other purification methods are possible. A purified material may contain less than about 50%, preferably less than about 75%, and most preferably less than about 90%, of the cellular components with which it was originally associated. The “substantially pure” indicates the highest degree of purity which can be achieved using conventional purification techniques known in the art.

In preferred embodiments, the terms “about” and “approximately” shall generally mean an acceptable degree of error for the quantity measured given the nature or precision of the measurements. Typical, exemplary degrees of error are within 20 percent (%), preferably within 10%, and more preferably within 5% of a given value or range of values. Alternatively, and particularly in biological systems, the terms “about” and “approximately” may mean values that are within an order of magnitude, preferably within 5-fold and more preferably within 2-fold of a given value. Numerical quantities given herein are approximate unless stated otherwise, meaning that the term “about” or “approximately” can be inferred when not expressly stated.

Molecular Physics

The term “sequence space” refers to the set of all possible sequences of residues for a polymer having a specified length. Thus, for example, the sequence space for a protein or polypeptide 300 amino acid residues in length is the group consisting of all sequences of 300 amino acid residues. Similarly, the sequences space of a nucleic acid 300 nucleotides in length is the group consisting of all sequences of 300 nucleotides, etc.

“Conformational energy” refers generally to the energy associated with a particular “conformation”, or three-dimensional structure, of a polymer, such as the energy associated with the conformation of a particular protein or nucleic acid. Interactions that tend to stabilize a macromolecule, such as a polymer (e.g., a protein or nucleic acid), have energies that are represented as negative energy values, whereas interactions that destabilize a polymer have positive energy values. Thus, the conformational energy for any stable polymer is quantitatively represented by a negative conformational energy value. Generally, the conformational energy for a particular polymer will be related to that polymer's stability. In particular, polymers and other macromolecules that have a lower (i.e., more negative) conformational energy are typically more stable, e.g., at higher temperatures (i.e., they have greater “thermal stability”). Accordingly, the conformational energy of a polymer may also be referred to as the polymer's “stabilization energy”.

Typically, the conformational energy is calculated using an energy “force-field” that calculates or estimates the energy contribution from various interactions which depend upon the conformation of a polymer. The force-field is comprised of terms that include the conformational energy of the alpha-carbon backbone, side chain—backbone interactions, and side chain—side chain interactions. Typically, interactions with the backbone or side chain include terms for bond rotation, bond torsion, and bond length. The backbone-side chain and side chain-side chain interactions include van der Waals interactions, hydrogen-bonding, electrostatics and solvation terms. Electrostatic interactions may include coulombic interactions, dipole interactions and quadrapole interactions). Other similar terms may also be included. Force-fields that may be used to determine the conformational energy for a polymer are well known in the art and include the CHARMM (see, Brooks et al, J. Comp. Chem. 1983,4:187-217; MacKerell et al., in The Encyclopedia of Computational Chemistry, Vol. 1:271-277, John Wiley & Sons, Chichester, 1998), AMBER (see, Cornell et al., J. Amer. Chem. Soc. 1995, 117:5179; Woods et al., J. Phys. Chem. 1995, 99:3832-3846; Weiner et al., J. Comp. Chem. 1986, 7:230; and Weiner et al., J. Amer. Chem. Soc. 1984, 106:765) and DREIDING (Mayo et al., J. Phys. Chem. 1990, 94-:8897) force-fields, to name a few.

In a preferred implementation, the hydrogen bonding and electrostatics terms are as described in Dahiyat & Mayo, Science 1997 278:82). The force field can also be described to include atomic conformational terms (bond angles, bond lengths, torsions), as in other references. See e.g., Nielsen J E, Andersen K V, Honig B, Hooft R W W, Klebe G, Vriend G, & Wade R C, “Improving macromolecular electrostatics calculations,” Protein Engineering, 12: 657662(1999); Stikoff D, Lockhart D J, Sharp K A & Honig B, “Calculation of electrostatic effects at the amino-terminus of an alpha-helix,” Biophys. J., 67: 2251-2260 (1994); Hendscb Z S, Tidor B, “Do salt bridges stabilize proteins—a continuum electrostatic analysis,” Protein Science, 3: 211-226 (1994); Schneider J P, Lear J D, DeGrado W F, “A designed buried salt bridge in a heterodimeric coil,” J. Am. Chem. Soc., 119: 5742-5743 (1997); Sidelar C V, Hendsch Z S, Tidor B, “Effects of salt bridges on protein structure and design,” Protein Science, 7: 1898-1914 (1998). Solvation terms could also be included. See e.g., Jackson S E, Moracci M, elMastry N, Johnson C M, Fersht A R, “Effect of Cavity-Creating Mutations in the Hydrophobic Core of Chymotrypsin Inhibitor 2,” Biochemistry, 32: 11259-11269 (1993); Eisenberg, D & McLachlan A D, “Solvation Energy in Protein Folding and Binding,” Nature, 319: 199-203 (1986); Street A G & Mayo S L, “Pairwise Calculation of Protein Solvent-Accessible Surface Areas,” Folding & Design, 3: 253-258 (1998); Eisenberg D & Wesson L, “Atomic solvation parameters applied to molecular dynamics of proteins in solution,” Protein Science, 1: 227-235 (1992); Gordon & Mayo, supra.

“Coupled residues” are residues in a polymer that interact, through any mechanism. The interaction between the two residues is therefore referred to as a “coupling interaction”. Coupled residues generally contribute to polymer fitness through the coupling interaction. Typically, the coupling interaction is a physical or chemical interaction, such as an electrostatic interaction, a van der Waals interaction, a hydrogen bonding interaction, or a combination thereof. As a result of the coupling interaction, changing the identity of either residue will affect the fitness of the polymer, particularly if the change disrupts the coupling interaction between the two residues. Coupling interaction may also be described by a distance parameter between residues in a polymer. If the residues are within a certain cutoff distance, they are considered interacting.

The term “fitness” is used to denote the level or degree to which a particular property or a particular combination of properties for a polymer (e.g., a biopolymer such as a protein or a nucleic acid) are optimized. In directed evolution methods of the invention, the fitness of a polymer is preferably determined by properties which a user wishes to improve. Thus, for example, the fitness of a protein may refer to the protein's thermal stability, catalytic activity, binding affinity, solubility (e.g., in aqueous or organic solvent), and the like. Other examples of fitness properties include enantioselectivity, activity towards non-natural substrates, and alternative catalytic mechanisms. Coupling interactions can be modeled as a way of evaluating or predicting fitness (stability). Fitness can be determined or evaluated experimentally or theoretically, e.g. computationally.

Preferably, the fitness is quantitated so that each polymer (e.g., each amino acid or nucleotide sequence) will have a particular “fitness value”. For example, the fitness of a protein may be the rate at which the polymer catalyzes a particular chemical reaction, or the protein's binding affinity for a ligand. In a particularly preferred embodiment, the fitness of a polymer refers to the conformational energy of the polymer and is calculated, e.g., using any method known in the art. See, e.g. Brooks B. R., Bruccoleri R E, Olafson, B D, States D J, Swaminathan S & Karplus M, “CHARMM: A Program for Macromolecular Energy, Minimization, and Dynamics Calculations,” J. Comp. Chem., 4: 187-217 (1983); Mayo S L, Olafson B D & Goddard W A G, “DREIDING: A Generic Force Field for Molecular Simulations,” J. Phys. Chem., 94: 8897-8909 (1990); Pabo C O & Suchanek E G, “Computer-Aided Model-Building Strategies for Protein Design,” Biochemistry, 25: 5987-5991 (1986), Lazar G A, Desjarlais J R & Handel T M, “De Novo Design of the Hydrophobic Core of Ubiquitin,” Protein Science, 6: 1167-1178 (1997); Lee C & Levitt M, “Accurate Prediction of the Stability and Activity Effects of Site Directed Mutagenesis on a Protein Core,” Nature, 352: 448-451 (1991); Colombo G & Merz K M, “Stability and Activity of Mesophilic Subtilisin E and Its Thermophilic Homolog: Insights from Molecular Dynamics Simulations,” J. Am. Chem. Soc., 121: 6895-6903 (1999); Weiner S J, Kollman P A, Case D A, Singh U C, Ghio C, Alagona G, Profeta S J, Weiner P, “A new force field for molecular mechanical simulation of nucleic acids and proteins,” J. Am. Chem. Soc., 106: 765-784 (1984). Generally, the fitness of a polymer is quantitated so that the fitness value increases as the property or combination of properties is optimized. For example, in embodiments where the thermal stability of a polymer is to be optimized (conformational energy is preferably decreased), the fitness value may be the negative conformationl energy; i.e., F=−E.

The term “fitness landscape” is used to describe the set of all fitness values belonging to all polymer sequences in a sequence space. Thus, for example, referring again to the sequence space for proteins 300 amino acid residues in length (i.e., the group consisting of all sequences of 300 amino acid residues), each polypeptide in the sequence space will have a particular fitness value that may (at least in theory) be calculated or measured (e.g., by screening each polypeptide to determine its fitness). The set of these fitness values is therefore the fitness landscape of the sequence space for proteins 300 amino acid residues in length. In many embodiments fitness values may vary considerably among individual sequences in a given sequence space. The fitness value for a given sequence may be higher or lower than other, similar sequences in the sequence space. These fitness values are therefore referred to as “local maxima” (or “local optima”) and “local minima”, respectively (e.g. when all single mutations lead to less-fit, i.e. less stable, mutants). Such a fitness landscape is described as “rugged” when it contains many local maxima and/or local minima in the fitness values. In the all representations of the fitness landscape, there is a “global optimum,” representing the single sequence with the highest fitness. If the highest fitness is degenerate (multiple sequence have the same fitness), then more than a single sequence can be the global optimum. A preferred objective of these directed evolution and computational design methods is generate sequences having fitness values greater than the fitness value(s) of the starting sequence or sequences. Still more preferably, the directed evolution and computational design methods of this invention generate sequences having fitness values as close to the global optimum as is possible.

The “fitness contribution” of a polymer residue refers to the level or extent f(iₐ) to which the residue iₐ, having an identity a, contributes to the total fitness of the polymer. Thus, for example, if changing or mutating a particular polymer residue will greatly decrease the polymer's fitness, that residue is said to have a high fitness contribution to the polymer. By contrast, typically some residues iₐ in a polymer may have a variety of possible identities a without affecting the polymer's fitness. Such residues, therefore have a low contribution to the polymer fitness.

The term “structural tolerance” is used to indicate the number of sequences or “sequence states” Ω in a particular sequence space that are compatible with a particular stabilization or conformational energy (generally referred to here as the “energy”). Usually the particular energy is the energy of a particular “parent” sequence (e.g., the sequence of a particular polypeptide or nucleic acid). A sequence state may be compatible with a particular energy if the sequence state's energy is equal to (or approximately equal to) the particular energy. In other embodiments, however, a sequence state may be compatible with a particular energy if the sequence state's energy is less than or equal to (or is less than or approximately equal to) the particular energy. Thus, in preferred embodiments of the invention, where a sequence space comprises mutants of a particular parent sequence, structurally tolerant mutants are those sequences in the sequence space (i.e., those mutants) having a stabilization or conformational energy that is compatible with the parent sequencers stabilization or conformational energy.

In preferred embodiments of the invention, the structural tolerance is determined or otherwise obtained or provided for each residue in a particular “parent” sequence (e g., for each amino acid residue of a protein sequence, or for each nucleotide of a nucleic acid sequence). In such embodiments, therefore, the structural tolerance of a particular residue is indicative of the number of compatible sequences having a mutation at that residue (i.e., where the identity of the corresponding residue in a compatible sequence is different from the residue's identity in the parent sequence).

In preferred embodiments, the structural tolerance of a polymer is measured by its “sequence entropy”. The sequence entropy S(E) for a particular polymer sequence (referred to herein as the “parent sequence”) is preferably obtained or provided by the relation

$$S ( E ) = {k}_{β} \cdot ln Ω = {\sum }_{t = 1}^{N} {S}_{t} \qquad ( Equation 1 )$$

where Ω is the number of polymer sequences in the sequence space containing the parent sequence which are compatible with a particular conformational energy E (preferably, the conformational energy of the parent sequence), and sᵢ is the “site entropy” (defined below) of residue i in the polymer sequence. See, Saven & Wolynes, J. Phys. Chem. B 1997, 101:8375. In this case k_{B} is a proportionality constant and may be selected, e.g., by a user, to have any value. In preferred embodiments this constant is equal to one (unity). In another preferred embodiment, k_{B} is the Boltzmann constant (e.g., about 1.38×10⁻²³ J/K).

The “site entropy” of a particular residue i in a particular polymer sequence (e.g., a parent sequence) is an indication or measurement of the number of compatible sequence states (i.e. sequence states that are compatible with a given conformational energy, preferably the conformational energy of the parent sequence) Ωᵢ⊂Ω that have a residue mutation or substitution at the residue corresponding to residue i in the parent sequence. Thus, the site entropy of a residue i is a measurement or indication of the extent or likelihood that a mutation at residue i will disrupt the three-dimensional structure and/or the “fitness” (defined supra) of the parent sequence.

In preferred embodiments, the site entropy sᵢ of residue i in a given polymer sequence is expressed as:

$${s}_{i} = - {k}_{B} {\sum }_{a}^{A} {p}_{i} ( a ) \cdot ln {p}_{i} ( a ) \qquad ( Equation 2 )$$

where A is the total number of substitutable groups (20 for amino acids), and k_{B} is the Boltzman constant (e.g., about 1.38×10⁻²³ J/K), or a dimensionless proportionality constant and is preferably chosen to be unity (i.e., k_{B}=1). The probability p,(a) is the probability “p” that amino acid residue “a” exists at residue “i.” These probabilities can be obtained by several methods. For instance, an energy cutoff can be imposed and all of the states (amino acids) with higher energies are assigned pᵢ(a)=0, whereas the remaining states are assigned equal probabilities.

In a preferred embodiment, all amino acids are effectively “varied” simultaneously at all residues. In an alternative embodiment, probabilities for pᵢ(a) can be determined by a Boltzmann weighting of the energies associated with mutating a residue to each state, while the remaining residues j (j≠i) retain their wild-type amino acid identities, i.e.:

$${p}_{i} ( a ) = \frac{{ⅇ}^{- β {E}_{i} ( a )}}{{\sum }_{{a}^{′}}^{A} {ⅇ}^{- β {E}_{i} ( {a}^{′} )}} . \qquad ( Equation 3 )$$

In this equation, Eᵢ(a) is the energy state a at residue i. For this embodiment, the mean field theory may be use to deconvolute the probabilities while effectively allowing all amino acids at all positions to vary.

The term “site entropy distribution” or “entropy distribution” refers to the distribution of site entropy values for a particular polymer sequence and may be represented, e.g., as a histogram of the polymer's site entropy values. Thus, for example, the site entropy distribution may provide, in certain embodiments, the probability P(sᵢ) that a residue of the polymer will have a particular site entropy value sᵢ. Equivalently, the site entropy distribution P(sᵢ) gives the fraction of residues in the polymer sequence that has a site entropy value sᵢ.

“Mean field theory” is a set of mathematical techniques for the theoretical treatment of systems undergoing phase transitions, using approximations. The idea of mean-field theory is to focus on one particular “tagged” particle in the system (in the case of proteins—one residue) and assume that the role of neighboring particles (residues) is to form an average energetic field which acts on the tagged particle (the specific amino acid at that residue). This is a useful technique to deconvolute the probability of sequence S_{A} into the product of the individual amino acid probabilities at each residue

$$P \{ {S}_{A} \} = {\prod }_{i = 1}^{N} p ( {i}_{a} ) . \qquad ( Equation 4 )$$

This method reduces the many-body problem (e.g., optimizing all the amino acids at all positions) to a one-body problem. Procedures of this type are not exact, but can be accurate and quite useful. See e.g., Chandler, D. Introduction to Modern Statistical Mechanics, Oxford University Press, Oxford, 1987.

“Dead-end elimination” (DEE) is a deterministic search algorithm that seeks to systematically eliminate bad rotamers and combinations of rotamers until a single solution remains. For example, amino acid residues can be modeled as rotamers that interact with a fixed backbone. The theoretical basis for DEE provides that, if the DEE search converges, the solution is the global minimum energy conformation (GMEC) with no uncertainty (Desmet et al., 1992).

Dead end elimination is based on the following concept. Consider two rotamers, iᵣ and iᵢ, at residue i, and the set of all other rotamer configurations {S} at all residues excluding i (of which rotamer jₛ is a member). If the pairwise energy contributed between iᵣ and jₛ is higher than the pairwise energy between iₜ and jₛ for all {S}, then rotamer iᵣ cannot exist in the global minimum energy conformation, and can be eliminated. This notion is expressed mathematically by the inequality.

$$E ( {i}_{r} ) + {\sum }_{j \neq i}^{N} E ( {i}_{r} , {j}_{s} ) > E ( {i}_{t} ) + {\sum }_{j \neq i}^{N} E ( {i}_{t} , {j}_{s} ) ∀ \{ S \} . \qquad ( Equation 5 )$$

If this expression is true, the single rotamer iᵣ can be eliminated (Desmet et al., 1992).

In this form, Equation 5 is not computationally tractable because, to make an elimination, it is required that the entire sequence (rotamer) space be enumerated. To simplify the problem, bounds implied by Equation 5 can be utilized:

$$E ( {i}_{r} ) + {\sum }_{j \neq i}^{N} {min}_{s} E ( {i}_{r} , {j}_{s} ) > E ( {i}_{t} ) + {\sum }_{j \neq i}^{N} {max}_{s} E ( {i}_{t} , {j}_{s} ) . \qquad ( Equation 6 )$$

Using an analogous argument, Equation (6) can be extended to the elimination of pairs of rotamers inconsistent with the GMEC. This is done by determining that a pair of rotamers iᵣ at residue i and jₛ at residue j, always contribute higher energies than rotamers iᵤ and jᵥ with all possible rotamer combinations {L}. Similar to Equation 6, the strict bound of this statement is given by:

$$ϵ ( {i}_{r} , {j}_{s} ) + {\sum }_{k \neq i , j}^{N} {min}_{t} ϵ ( {i}_{r} , {j}_{s} , {k}_{t} ) > ϵ ( {i}_{u} , {j}_{v} ) + {\sum }_{k \neq i , j}^{N} {max}_{t} ϵ ( {i}_{u} , {j}_{v} , {k}_{i} ) \qquad ( Equation 7 )$$

where ε is the combined energies for rotamer pairs

ε(iᵣ,jₛ)=E(iᵣ)+E(jₛ)+E(iᵣ,jₛ) (Equation 8)

and

ε(iᵣ,jₛ,kₜ)=E(iᵣ,kₜ)+E(jₛ,kₜ) (Equation 9).

This leads to the doubles elimination of the pair of rotamers iᵣ and jₛ, but does not eliminate the individual rotamers completely as either could exist independently in the GMEC. The doubles elimination step reduces the number of possible pairs (reduces S) that need to be evaluated in the right-hand side of Equation 6, allowing more rotamers to be individually eliminated.

The singles and doubles criteria presented by Desmet et al. fail to discover special conditions that lead to the determination of more dead-ending rotamers For instance, it is possible that the energy contribution of rotamer iₜ is always lower than iᵣ without the maximum of iₜ being below the minimum of iᵣ. To address this problem, Goldstein 1994 presented a modification of the criteria that determines if the energy profiles of two rotamers cross. If they do not, the higher energy rotamer can be determined to be dead-ending. The doubles calculation significantly more computational time than the singles calculation. To accelerate the process, other computational methods have been developed to predict the doubles calculations that will be the most productive (Gordon & Mayo, 1998). These kinds of modifications, collectively referred to as fast doubles, significantly improved the speed and effectiveness of DEE.

Several other modifications also enhance DEE. Rotamers from multiple residues can be combined into so-called super-rotamers to prompt further eliminations (Desmet et al., 1994; Goldstein, 1994). This has the advantage of eliminating multiple rotamers in a single step. In addition, it has been shown that “splitting” the conformational space between rotamers improves the efficiency of DEE (Pierce et al., 2000). Splitting handles the following special case. Consider rotamer iᵣ. If a rotamer iₜ₁ contributes a lower energy than iᵣ for a portion of the conformational space, and a rotamer iₜ₂ has a lower energy than iᵣ for the remaining fraction, then iᵣ can be eliminated. This case would not be detected by the less sensitive Desmet or Goldstein criteria. In the preferred implementations of the invention as described herein, all of the described enhancements to DEE were used.

For further discussion of these methods see, Goldstein, R. F. (1994), Efficient rotamer elimination applied to protein side-chains and related spin glasses, Biophysical Journal 66, 1335-1340; Desmet, J., De Maeyer, M., Hazes, B. & Lasters, I. (1992), The dead-end elimination theorem and its use in protein side-chain positioning. Nature 356,539-542; Desmet, J., De Maeyer, M. & Lasters, I. (1994), In The Protein Folding Problem and Tertiary Structure Prediction (Jr., K. M. & Grand, S. L., eds.), pp. 307-337 (Birkhauser, Boston); De Maeyer, M., Desmet, J. & Lasters, I. (1997), All in one: a highly detailed rotamer library improves both accuracy and speed in the modeling of side chains by dead-end elimination, Folding & Design 2, 53-66, Gordon, D. B. & Mayo, S. L. (1998), Radical performance enhancements for combinatorial optimization algorithms based on the dead-end elimination theorem, Journal of Computational Chemistry 19, 1505-1514; Pierce, N. A., Spriet, J. A., Desmet, J., Mayo, S. L., (2000), Conformational splitting: A more powerful criterion for dead-end elimination; Journal of Computational Chemistry 21, 999-1009.

5.2. General Methods

In accordance with the invention, there may be employed conventional molecular biology, microbiology and recombinant DNA techniques within the skill of the art. Such techniques are explained fully in the literature. See, for example, Sambrook, Fitsch & Maniatis, Molecular Cloning: A Laboratory Manual, Second Edition (1989) Cold Spring Harbor Laboratory Press, Cold Spring Harbor, N.Y. (referred to herein as “Sambrook et al., 1989”); DNA Cloning: A Practical Approach, Volumes I and II (D. N. Glover ed. 1985); Oligonucleotide Synthesis (M. J. Gait ed. 1984); Nucleic Acid Hybridization (B. D. Hames & S. J. Higgins, eds. 1984); Animal Cell Culture (R. I. Freshney, ed. 1986); Immobilized Cells and Enzymes (IRL Press, 1986); B. E. Perbal, A Practical Guide to Molecular Cloning (1984); F. M. Ausubel et al. (eds.), Current Protocols in Molecular Biology, John Wiley & Sons, Inc. (1994).

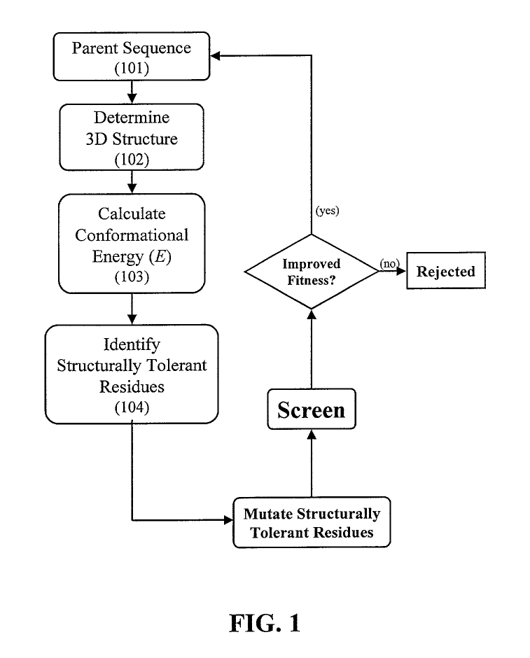

FIG. 1 provides a flow diagram illustrating a general, exemplary embodiment of the methods used in this invention. In particular, the flow diagram in FIG. 1 as well as the other examples presented in Section 6, infra, describe preferred embodiments where the methods are used in directed evolution of a protein or other polypeptide. Those skilled in the art can readily appreciate, however, that the methods illustrated by these examples and throughout this specification may be used to modify any polymer. Indeed, any molecule composed of a sequence or series of discrete residues can be optimized according to these methods. Thus, the methods of the invention may also be used, e.g., for directed evolution of nucleic acids (including directed evolution of DNA or RNA). A person skilled in the relevant art(s) may readily modify the methods for use with such other polymers using only what is taught in this application coupled with routine methods, already known in the art, for synthesizing, modifying and/or screening other polymers.

The Parent Sequence

The method shown in FIG. 1 begins with the selection of a “parent” amino acid (or other polymer) sequence (101). The parent sequence may be any amino acid sequence and may or may not correspond to a naturally occurring polypeptide. However, in preferred embodiments the parent sequence will be the sequence for a protein, or other polypeptide, that is expressed by a cell. Preferably, the parent sequence is also the sequence for a protein that has some level or degree of activity or function (e.g., catalytic activity, binding affinity, solubility, thermal stability, etc.) to be optimized. The methods of the invention may then be used, e.g., to optimize the activity or function of the parent sequence and/or to optimize the activity in altered conditions. For example, in one embodiment the parent sequence may be a protein having a particular catalytic or other activity, and the invention may be used to identify sequences having the same activity but under different (generally more extreme) conditions such as conditions of temperature or of solvent (including, for example, solvent polarity, salt conditions, acidity, alkalinity, etc.). In another embodiment, the parent sequence may have a particular level or amount of activity (e.g., catalytic activity, binding affinity, etc.), and the directed evolution methods of the invention may be used to identify sequences having improved levels or amounts of that same activity (e.g., higher binding affinity or increased catalytic rate).

A Generic Statistical Model of Coupling Interactions

Preferably, the fitness value of the parent sequence is determined (e.g., calculated) or otherwise obtained or provided from an expression that comprises a first term f(iₐ) for the uncoupled contribution of each residue iₐ to the fitness, and a second term f(iₐ,jₐ) for the contribution made by coupling interactions between residues iₐ and jₐ; i.e., of the general form

$$F = {\sum }_{i = 1}^{N} f ( {i}_{a} ) + b {\sum }_{i = 1}^{N - 1} {\sum }_{j > i}^{N} f ( {i}_{a} , {j}_{a} ) {λ}_{if} \qquad ( Equation 10 )$$

where N is the number of residues and the constant b is the relative strength of the coupled and uncoupled interactions. λ_{g}=1 if there is a coupling interaction between residues iₐ and jₐ; otherwise λᵢⱼ=0. Similar fitness approximations that use one- and two-body terms are known in the art and have been used previously to model thermal stability (see, e.g., Dahiyat & Mayo, Science 1997, 278:82; Malakaukas & Mayo, Nature Structural Biology 1998, 5:470; Saven & Wolynes, J. Phys. Chem. B. 1997,101:8375; Abkevich et al., J. Mol. Biol. 1995, 252:460; Li et al., Science 1996, 273:666).

Accordingly, in preferred embodiments where the fitness is provided by the negative value of the conformational energy; i.e., where F=−E, the conformational energy may be obtained or provided from an expression of the form

$$E = {\sum }_{i = 1}^{N} e ( i ) + {\sum }_{i = 1}^{N - 1} {\sum }_{j > 1}^{N} e ( i , j ) \qquad ( Equation 11 )$$

where e(iᵣ) denotes interactions between components (typically atoms or functional groups, such as methyl-groups, ethyl-groups, hydroxyl-groups, etc.) of the same residue i (i.e., intra-residue interactions) that contribute to the conformational energy, and e(i, j) denotes interactions between components of different residues i and j (i.e., inter-residue interactions) that contribute to the conformational energy, such as (but not limited to) van der Waals, electrostatic, and hydrogen bonding interactions between different residues.

Determining Three-dimensional Structure

In preferred embodiments, the structure or conformation of the parent sequence is obtained or otherwise provided. (See FIG. 1, step 102). In many preferred embodiments, and particularly in embodiments where the parent sequence is the sequence for a known protein or nucleic acid, the structure or conformation of the parent sequence will be known and can be obtained from any of a variety of resources (for a review, see Hogue et al., Methods Biochem. Anal. 1998, 39:46-73). For example, and not by way of limitation, the Protein Data Bank (PDB) (Berman et al., Nucl. Acids Res. 2000, 28:235-242) is a public repository of three-dimensional structures for a large number of macromolecules, including the structures of many proteins, nucleic acids and other biopolymers.

Alternatively, in many embodiments the structure of a polymer (e.g., protein) sequence that is similar or homologous to the parent sequence will be known. In such instances, it is expected that the conformation of the parent sequence will be similar to the known structure of the homologous polymer. The known structure may, therefore, be used as the structure for the parent sequence or, more preferably, may be used to predict the structure of the parent sequence (i.e., in “homology modeling”). As a particular example, the Molecular Modeling Database (MMDB) (see, Wang et al., Nucl. Acids Res. 2000, 28:243-245; Marchler-Bauer et al., Nucl. Acids Res. 1999,27:240-243) provides search engines that may be used to identify proteins and/or nucleic acids that are similar or homologous to a parent sequence (referred to as “neighboring” sequences in the MMDB), including neighboring sequences whose three-dimensional structures are known. The database further provides links to the known structures along with alignment and visualization tools whereby the homologous and parent sequences may be compared and a structure may be obtained for the parent sequence based on such sequence alignments and known structures.

In other embodiments, where the structure for a particular parent sequence may not be known or available, it is typically possible to determine the structure using routine experimental techniques (for example, X-ray crystallography and Nuclear Magnetic Resonance (NMR) spectroscopy) and without undue experimentation. See, e.g., NMR of Macromolecules: A Practical Approach, G. C. K. Roberts, Ed., Oxford University Press Inc., New York (1993); Ishima R, Torchia D A, “Protein Dynamics from NMR,” Nat Struct Biol, 7: 740-743 (2000); Gardner K H, Kay L E, “The use of H-2, C-13, N-15 multidimensional NMR to study the structure and dynamics of proteins,” Annu. Rev. Bioph. Biom., 27: 357-406 (1998); Kay LE, “NMR methods for the study of protein structure and dynamics,” Biochern Cell Biol, 75: 1-15 (1997); Dayie K T, Wagner G, Lefevre J F, “Theory and practice of nuclear spin relaxation in proteins,” Annu Rev Phys Chem, 47: 243-282 (1996); Wuthrich K, “NMR—This and other methods for protein and nucleic-acid structure determination,” Acta Cyrstallogr. D, 51: 249-270 (1995); Kahn R, Carpentier P, Berthet-Colominas C, et al., “Feasibility and review of anomalous X-ray diffraction at long wavelengths in materials research and protein crystallography,” J. Synchrotron Radiat., 7: 131-138 (2000); Oakley A J, Wilce M C J, “Macromolecular cyrstallography as a tool for investigating drug, enzyme and receptor interactions,” Clin. Exp. Pharmacol. P.,27:145-151 (2000); Fourme R, Shepard W, Schiltz M, et al., “Better structures from better data through better methods: a review of developments in de novo macromolecular phasing techniques and associated instrumentation at LURE,” J. Synchrotron Radiat., 6: 834-844 (1999).

Alternatively, and in less preferable embodiments, the three-dimensional structure of a parent sequence may be calculated from the sequence itself and using ab initio molecular modeling techniques already known in the art. See e.g., Smith T F, LoConte L, Bienkowska J, et al., “Current limitations to protein threading approaches,” J. Comput. Biol., 4: 217-225 (1997); Eisenhaber F, Frommel C, Argos P, “Prediction of secondary structural content of proteins from their amino acid composition alone 2. The paradox with secondary structural class,” Proteins, 24: 169-179 (1996); Bohm G, “New approaches in molecular structure prediction,” Biophys Chem., 59: 1-32 (1996); Fetrow J S, Bryant S H, “New programs for protein tertiary structure prediction,” BioTechnol., 11: 479-484, (1993); Swindells M B, Thorton J M, “Structure prediction and modeling,” Curr. Opin. Biotech., 2: 512-519 (1991); Levitt M, Gerstein M, Huang E, et al., “Protein folding: The endgame,” Annu. Rev. Biochem., 66: 549-579 (1997). Eisenhaber F., Persson B., Argos P., “Protein-structure prediction—recognition of primary, secondary, and tertiary structural features from amino-acid-sequence,” Crit Rev Biochem Mol, 30:1-94(1995); Xia Y, Huang E S, Levitt M, et al., “Ab initio construction of protein tertiary structures using a hierarchical approach,” J. Mol. Biol., 300:171-185 (2000); Jones D T, “Protein structure prediction in the post genomicera,” Curr Opin Struc Biol, 10: 371-379 (2000). Three-dimensional structures obtained from ab initio modeling are typically less reliable than structures obtained using empirical (e.g., NMR spectroscopy or X-ray crystallography) or semi-empirical (e.g., homology modeling) techniques. However, such structures will generally be of sufficient quality, although less preferred, for use in the methods of this invention.

Determining Conformational Energy

Once a three-dimensional structure has been obtained or otherwise provided for the parent sequence, a fitness value for the parent may be optionally obtained by calculating or determining the “conformational energy” or “energy” E of the parent structure (103). In particular and without being limited to any particular theory or mechanism of action, sequences that have a lower (i.e., more negative) conformational energy are typically expected to be more stable and therefore more “fit” than are sequences having higher (i.e., less negative) conformation energy. Thus, the fitness of a sequence is preferably related to its negative conformational energy; i.e., F=−E.

Typically, the conformational energy is calculated ab initio from the conformation determined in step 102, discussed above, and using an empirical or semi-empirical force field such as CHARM (Brooks et al., J. Comp. Chem. 1983, 4:187-217; MacKerell et al., in The Encyclopedia of Computational Chemistry, Vol. 1:271-277, John Wiley & Sons, Chichester, 1998) AMBER (see, Cornell et al., J. Amer Chem. Soc. 1995, 117:5179; Woods et al., J. Phys. Chem. 1995,99:3832-3846; Weineretal., J. Comp. Chem. 1986,7:230; and Weiner et al., J. Amer. Chem. Soc. 1984, 106:765) and DREIDING (Mayo et al., J. Phys. Chem. 1990, 94:8897) to name a few. These and other such force-fields comprise a number of potential functions and parameters for at least approximate contributions of various interactions within a macromolecule, such as electrostatic interactions, van der Waals interactions, hydrogen bonding interactions, etc.

In alternative embodiments, the fitness expression for a polymer may include additional terms for coupling interactions beyond pairwise coupling contributions. For example, Equation 4, above, may be expanded to include an additional term f(iₐ, jₐ, kₐ) for contributions made by coupling interactions between triplets of residues iₐ,jₐ and kₐ. Indeed, expressions for the fitness of a sequence may comprise terms for coupling interactions between any multiple of residues. However, the difficulty of calculating the fitness value of a sequence increases exponentially as additional coupling terms are included. In preferred embodiments, therefore, where the fitness value of a sequence is calculated from the negative value of its conformational energy, the energy force field may be which is expanded beyond the pairwise form to include multi-body energy terms such as, but not limited to, buried hydrophobic surface area and more complicated electrostatic interactions (e.g., electrostatic dipole and/or electrostatic quadropole interactions).

These concepts can be illustrated by the following chart, showing a hypothetical calculation using representative (not actual) energy and temperature values. For a single residue, the energy of each amino acid substitution is listed in the chart. Each column after the energy is the list of probabilities associated with a temperature. For each temperature, the entropy of this position and the mean-field energy are given. As the probability is decreased, the mean-field energy decreases (as well as the entropy). Referring for example to the probabilities at T=10, all of the sequences consistent with energy E=−21.3 are effectively shown. For example, 51% of the sequences at this energy contain Val at this residue and 3% contain Gly.

Energy Chart for Hypothetical Residue A

| | | Temperature | | | |
|---|---|---|---|---|---|
| | energy | 1000 | 100 | 10 | 1 |
| ALA | −10 | 0.05 | 0.07 | 0.07 | 0.00 |
| ARG | 15 | 0.05 | 0.05 | 0.01 | 0.00 |
| ASN | −5 | 0.05 | 0.07 | 0.04 | 0.00 |
| ASP | 25 | 0.05 | 0.05 | 0.00 | 0.00 |
| CYS | 3 | 0.05 | 0.06 | 0.02 | 0.00 |
| GLN | 4 | 0.05 | 0.06 | 0.02 | 0.00 |
| GLU | −22 | 0.05 | 0.08 | 0.23 | 0.00 |
| GLY | −1 | 0.05 | 0.06 | 0.03 | 0.00 |
| HIS | 14 | 0.05 | 0.05 | 0.01 | 0.00 |
| ILE | 100 | 0.05 | 0.02 | 0.00 | 0.00 |
| LEU | 125 | 0.05 | 0.02 | 0.00 | 0.00 |
| LYS | 53 | 0.05 | 0.04 | 0.00 | 0.00 |
| MET | 29 | 0.05 | 0.05 | 0.00 | 0.00 |
| PHE | −10 | 0.05 | 0.07 | 0.07 | 0.00 |
| PRO | 150 | 0.04 | 0.01 | 0.00 | 0.00 |
| SER | 20 | 0.05 | 0.05 | 0.00 | 0.00 |
| THR | 93 | 0.05 | 0.02 | 0.00 | 0.00 |
| TRP | 72 | 0.05 | 0.03 | 0.00 | 0.00 |
| TYR | 19 | 0.05 | 0.05 | 0.00 | 0.00 |
| VAL | −30 | 0.05 | 0.08 | 0.51 | 1.00 |

| | | | | |
|---|---|---|---|---|
| entropy | 2.99 | 2.91 | 1.55 | 0.00 |
| mean-field energy | 29.84 | 13.57 | −21.30 | −30.00 |

This chart is an example of how the calculations of the invention can be done. In an actual calculation, each amino acid has a set of rotamers, each of which has an associated energy (and therefore a probability). The amino acid probability, which is used to calculate the entropy, is the sum of the rotamer probabilities. In addition, the energies presented in the simplified example above are for single residues. This does not account for coupling between amino acids.

The addition of coupling has the following affect on the residue presented above (Residue A). Imagine a second residue in the protein (Residue B). If Residue A is GLU, then Residue B can be TRP or ALA. If Residue A is VAL, then Residue B can only be ALA. At T=10, Residue A is effectively 51% VAL and 23% GLU. At this “temperature” ALA is slightly more favored at Residue B. However, at T=1, Residue A is 100% VAL, and the only amino acid that can exist at Residue B is ALA. In effect, the restriction of the amino acid identity at Residue A restricts the amino acid identity at Residue B. The mean-field calculation simultaneously handles all of these restrictions (which arise due to interactions between amino acids).

Structural Tolerance

Once the three-dimensional structure (102) and/or the conformational energy (103) have been obtained or provided for a particular parent sequence, the structural tolerance (104) can be readily determined for each residue (e.g., for each amino acid or nucleic acid residue) of that parent sequence using the methods provided herein. In particular and as demonstrated in the example shown in Section 6.1, infra, mutations that improve the fitness of a particular polymer are most likely to occur at uncoupled residues or at residues that are only weakly coupled. This is particularly true in preferred embodiments of the invention where the parent sequence is the sequence of a polymer, such as a protein or nucleic acid, that has a relatively high level of fitness. Accordingly, in preferred embodiments the structural tolerance of a residue is determined by evaluating the level or degree of coupling interactions that residue has with other residues of the polymer. Counting the number of coupling interactions between each residue is a simple means of estimating the structure tolerance of a residue. For example, a residue that has many coupling interactions between itself and the remaining structure is intolerant and a residue that has few coupling interactions between itself and the remaining structure is tolerant.

In a particularly preferred embodiment, the structural tolerance of a residue i is provided or determined by obtaining or determining the “site entropy” sᵢ of that residue. In such embodiments, the site entropy sᵢ is related to the number of sequences Ωᵢ in the sequence space that are compatible with conformational energy E of the parent sequence and have a mutation (i.e., substitution) at residue i. Residues that have higher site entropy values are residues where more mutations may be made which are compatible with the conformational energy of a parent sequence. Accordingly, the site entropy is particularly useful as a measurement of a residue's structural tolerance for mutations. In general embodiments, the site entropy may be provided by any relationship where sᵢ increases with Ωᵢ.

In various embodiments, the site entropy sᵢ of a residue may be calculated or obtained by identifying all compatible sequences Ω in the sequence space, and identifying, among these compatible sequences, those that have a mutation or substitutions at residue i (i.e., those compatible sequence where the residue at position i has a different identity than in the parent sequence). In these embodiments, the sequences will include compatible sequences in the sequence space which have mutations or substitutions at one or more other residues j≠i in addition to a mutation or substitution at residue i. Practically speaking, however, it will be computationally intractable to identify all possible sequences Ω in a sequence space that are compatible with the conformational energy of a particular parent sequence. Accordingly, the invention also provides embodiments where the site entropy sᵢ of a residue may be calculated or obtained by identifying compatible sequences that are identical to the parent except for a single mutation or substitution at residue i. In other embodiments, the methods of the invention may involve identifying and/or determining the number of compatible structures having the same sequence as the parent and having multiple residues where amino acid substitutions are allowed simultaneously.

With reference to the example of FIG. 1, and not by way of limitation, a skilled artisan may readily determine the conformational energy of a mutant sequence that differs from the parent by a single mutation or substitution at residue i, using the three dimensional structure provided for the parent sequence (102). In preferred embodiments, the conformational energy for all possible mutations and/or substitutions of the residue i is modelled (e.g., for all possible amino acid residue substitutions or for all possible nucleotide substitutions). Those single residue mutations and/or substitutions that have a conformational energy which is compatible with the conformational energy provided for the parent (103) are identified, and are used to calculate or determine the site entropy value sᵢ.

The invention also provides embodiments where the site entropy sᵢ for each residue in a polymer sequence is iteratively calculated. As a particular illustration of such embodiments, Examples 6.2 demonstrates exemplary calculations of site entropy values using mean-field theory to identify sequences that are compatible with the conformational energy of certain proteins that are used (in that example) as parent sequences. Conformational energies in these particularly preferred embodiments may be calculated, for example, using an energy force-field that models interactions of amino acid residues as rotamers interacting with a fixed backbone.

The mean field calculation effectively varies all amino acids of a parent protein simultaneously, across a fitness range that is modeled according to relative conformation energies across a corresponding “sequence temperature” range. The sequence temperature is a convenient term for representing relative energies; it is not a physical or measured temperature in the sense of degrees Kelvin or Centigrade Conceptually, low fitness corresponds to high sequence temperatures, and high fitness corresponds to low sequence temperatures. At high enough temperatures the fitness is effectively zero, all the amino acids are equally probable, all of the site entropies are at a maximum, and no mutations (e.g. amino acid deletions, additions or substitutions) are identified or distinguished as more or less structurally tolerant than others. As the temperature is lowered, fitness increases, and some of the probabilities associated with particular amino acids appearing at particular locations will change. The mean field equations are iterated for self-consistency across the range of temperatures used.

For example, a hypothetical polypeptide having amino acid “X” at position 25 may be substituted at that position by any other amino acid. In a mean field model, the probability of finding alanine at position 25 (according to the conformational energies at a first (relatively high) temperature may be 0.1, whereas the probability associated with serine may be 0.9. According to the invention, this means that serine has a lower energy than alanine at position 25, and serine is expected to be “better” for the fitness of the polypeptide than alanine. Likewise, if the amino acid “Y” at position 30 of this polypeptide interacts with the amino acid at position 25, then amino acids that interact more favorably with serine will be preferred as amino acid Y at position 30. This is an example of “coupled residues” as discussed herein. Mutations which preserve, or tend not to disrupt, the coupling interaction between amino acid X²⁵ and amino acid y³⁰ are expected to provide a more structurally tolerant hybrid polypeptide. This in turn is more likely to result in a functional mutant which may have improved properties. As the temperature in the model is lowered (the fitness is increased), the probabilities associated with each amino acid at each position become more skewed, with a few amino acids having high probabilities and many having lower probabilties. According to the Equation 2 for site entropy, the entropies associated with each amino acid at each position decrease. According to the invention, those that decrease the fastest correlate with the most highly coupled residues.

Embodiments such as the mean-field theory embodiment demonstrated in Section 6.2 are particularly preferred since these embodiments effectively vary all residues in a given polymer sequence simultaneously. However, in an alternative aspect of such embodiments, particular residues (e.g., one or more, two or more, three or more, four or more, five or more, ten or more, etc.) of a polymer sequence may be selected (e.g., by a user) and the calculations demonstrated in Section 6.2 may be performed varying only the selected residues. In such aspects of these embodiments, only the probability distribution of the selected residue(s) is (are) determined. In another aspect of these embodiments, therefore, first one residue is picked and its probability distribution is calculated (e.g., according to the mean field theory described in Example 6.2), and a second residue is then picked and its probability distribution is calculated, etc.; until site entropies are calculated for a plurality of residues in the polymer sequence (preferably for all residues in the polymer sequence). Stated another way, one residue is picked to vary (in composition and conformation) while holding all of the other residues constant (e.g. in their wild-type state). Calculations like those above are done, but only the probability distribution for the picked residue is determined when the temperature is decreased (the site entropy is calculated only for that residue). Then, a new residue is picked, and the process is repeated as desired, typically until the site entropies for all of the positions have been determined.

Other embodiments within the spirit of the invention will also be apparent to those skilled in the art. For example, and not by way of limitation, stochastic algorithms such as Monte Carlo algorithms may be used to determine conformational energies for a large number of sequences folded into a fixed backbone (preferably, one that corresponds to the conformation of the backbone in a parent sequence), and to identify those sequences which are compatible with (e.g. less than or approximately equal to) the conformational energy of the parent sequence, this giving an estimate of sᵢ. See e.g., Desjarlais J R & Clarke N D, “Computer search algorithms in protein modification and design,” Curr. Opin. Struct. Biol., 8: 471-475 (1998); Sasai M., “Conformation, energy, and folding ability of selected amino acid sequences,” Proc. Natl. Acad. Sci. USA, 92: 8438-8442 (1995); Dahiyat B I, & Mayo S L, “Probing the role of packing specificity in protein design,” Proc. Natl. Acad. Sci. USA, 94: 10172-10177 (1997); Godzik A., “In search of the ideal protein sequence,” Protein Engineering, 8: 409-416 (1995). Moreover, interactions between residues of a polymer such as a protein may be modeled using, e.g., a continuous side chain model instead of a rotamer model. Compatible sequences may be identified in such a continuous side chain model, e.g., with molecular dynamics simulations.

In still other embodiments, a parent sequence may be compared to one or more (preferably to a plurality of ) polymer sequences to identify homologous sequences. For example, in one preferred embodiment, the parent sequence (for example, a particular protein sequence) may be aligned with a plurality of other sequences (e.g., from a database of naturally occurring protein or other polymer sequences, such as the GenBank, SWISPROT or EMBL database) to identify homologous sequences. Sequences that have a certain level of sequence similarity may also be compared. Generally, the level of sequence similarity is a threshold level or percentage of sequence homology (or sequence identity) that may be selected by a user. For example, preferred levels of sequence homology (or identity) are at least 70%, at least 75%, at least 80%, at least 85%, at least 90%, at least 95% or at least 99%. A variety of methods and algorithms are known in the art for aligning polymer sequences and/or determining their levels of sequence similarity. Any of these methods and algorithms may be used in connection with this invention. Exemplary algorithms include, but are not limited to, the BLAST family of algorithms, FASTA, MEGALIGN and CLUSTAL.

Once homologous sequences have been identified and/or aligned with the parent sequence, the site entropy of one or more particular residues in the parent -sequence may be determined or estimated from the number of homologous or aligned sequences in which the particular residue is mutated. As used to describe this invention, a homologous or aligned polymer sequence is said to have a mutation at a particular residue if, in an alignment of the particular polymer sequence (e.g., from the alignment algorithm used to identify a homologous sequence or sequences), the residue in the homologous sequence which that aligns with the particular residue in the parent sequence is different (i.e., has a different identity) from the particular residue. The probabilities required to determine the site entropy (e.g. Equation 2) can be calculated by the relative number of times each amino acid appears at each residue in the alignment.

In other embodiments, the structural tolerance of residues in a polymer may be determined indirectly through other parameters that arc related to or that correlate with structural tolerance. For example, in embodiments where an X-ray structure of the parent sequence is available or may be obtained, B-values of individual residues may correlate with the individual residues' structural tolerance and can be used as indicators thereof. (Baase et al., pages 297-311 in Simplicity and Complexity in Proteins and Nucleic Acids, Fraenfelder et al., eds., Dahlem University Press, 1999). Similarly, if an ensemble of structures is available or may be readily determined (e.g., by NMR) for the parent sequence, per residue root mean squared (rms) deviation values from the ensemble may also be used as indicators of structural tolerance.

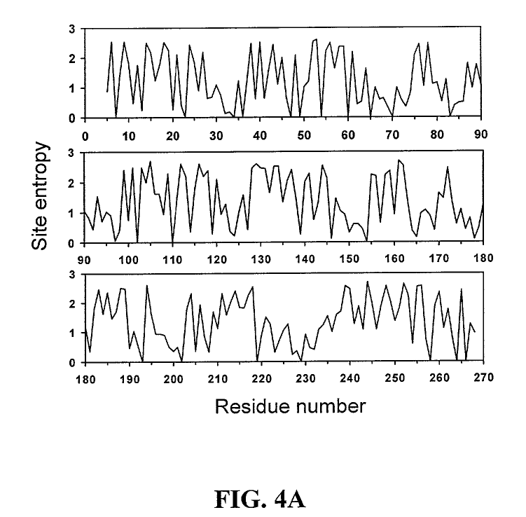

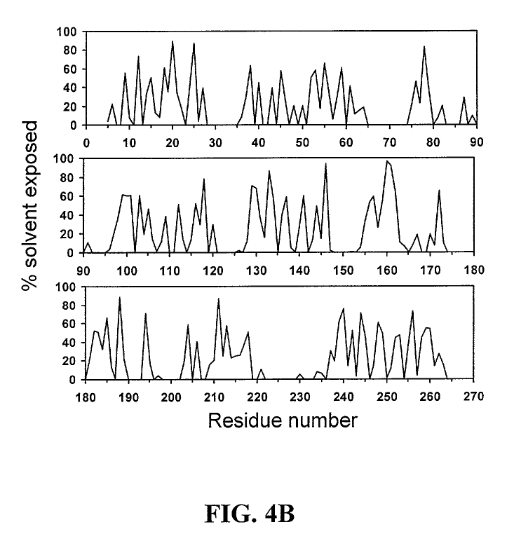

As yet another example, Section 6.3 demonstrates that site entropy values for residues (e.g., as determined according to the method demonstrated in Section 6.2) correlate, at least to some degree, with the solvent accessibility of each residue in the parent sequence (e.g., in a protein). See, Tables 4A and 4B, and FIGS. 4A and 4B. Solvent accessibility for each residue was calculated using the Lee and Richards definition of solvent accessible surface area (Lee, B. & Richards, F. M. (1971) J. Mol. Biol. 55, 379) where 1.4 Å was used as the radius for water. Accordingly, the level or extent to which a residue in a polymer is accessible to solvent may also be used to indicate structural tolerance.

5.3. Directed Evolution

The methods described in Section 5.2, supra, are particularly useful for directed evolution experiments, e.g., to obtain proteins, nucleic acids or other polymers having one or more desirable properties. Accordingly, the invention also provides methods, including methods of directed evolution, for obtaining polymers that have one or more improved properties. The improved properties include any property or combination of properties that can be detected by a user and include, for example, properties of catalytic activity (for example, increased rates of catalysis), properties of stability (for example, increased thermal stability) or properties of binding affinity (for example, increased affinity for a particular ligand or substrate) and properties of binding specificity (including stereo- or enantio-selectivity; i.e., the specificity with which a polymer binds to one stereo-isomer or enantiomer of a compound compared to another stereo-isomer) to name a few.

In general, directed evolution methods comprise selecting at least one polymer sequence (i.e., a “parent” sequence). Usually, the polymer sequence is the sequence for a polymer (e.g., a nucleic acid or a polypeptide) that has a particular property or properties of interest. For example, the particular property of the parent may be a particular catalytic activity, binding to a particular substrate or ligand, thermal stability or a combination thereof. Preferably the property is one that can be readily determined or evaluated by a screening assay, e.g. a high throughput screen. One or more residues of the parent polymer sequence is selected or targeted for mutation. In traditional methods for directed evolution, e.g. error-prone PCR, point mutagenesis is applied across an entire gene. This is a random process, and mutations appear at random sites. However, in the methods of the invention, specific residues in the parent sequence which are structurally tolerant are selected. The structurally tolerant residues may be identified, for example, according to the analytical methods described supra (see, Section 5.2). The eliminates or reduces the random mutagenesis of known methods, and provides a more targeted approach with improved efficiency.

One or more, and preferably a plurality of mutant polymer sequences may then be generated based on the parent sequence. In particular, the directed evolution methods of the invention preferably generate a plurality of mutants which are identical to the parent sequence except that one or more structurally tolerant residues are mutated. Polymers having the mutant sequences may then be generated using polymer synthesis and or recombinant technologies well known in the art, and the polymers having these mutant sequences are then preferably screened for the one or more properties of interest.

In preferred embodiments, methods of directed evolution may be iteratively repeated to generate and identify polymers where one or more properties of interest progressively improve with each iteration. Accordingly, in a preferred embodiment, one or more of the selected polymers may be selected as a new parent sequence, for use in a next round of iteration in the directed evolution method. Structurally tolerant residues of the new parent sequence may then be selected, and a second generation of mutants can be generated and screened as described above. Improved mutants may also be recombined if desired, using conventional genetic engineering techniques or by DNA shuffling to obtain further variations and improvements (see, for example, the Stemmer references, supra). These processes may be repeated as desired, to obtain successive generations of mutants.

Methods for the directed evolution of polymers such as nucleic acids and polypeptides are well known in the art. See, for example, Dube et al., Gene 1993, 137:41; Moore & Arnold, Nature Biotechnology 1996, 14:458; Joo et al., Nature 1999, 399:670; Zhao & Arnold, Protein Engineering 1999, 12:47; Skandalis et al., Chem. Biol. 1997, 4:889-898; Nikolova et al., Proc Natl. Acad. Sci. U.S.A. 1998, 95:14675; Miyazaki & Arnold, J. Molecular Evolution 1999, 49:716. See, also, U.S. Pat. Nos. 5,741,691 and 5,811,238; International Patent Applications WO 98/42832, WO 95/22625, WO 97/20078, WO 95/41653, and U.S. Pat. Nos. 5,605,793 and 5,830,721. Generally, such methods work by selecting a parent sequence, typically a particular protein, and generating large numbers of mutants, for example by error prone PCR of a gene encoding the selected protein (see, e.g., Dube et al., Gene 1993, 137:41; Moore & Arnold, Nature Biotechnology 1996, 14:458; Joo et al., Nature 1999, 399:670; Zhao & Arnold, Protein Engineering 1999, 12:47). The mutants are then tested, preferably in a screening assay, to identify mutants that actually have an improved property detected in the assay (for example, increased catalytic activity, or stronger binding to a ligand or substrate). These mutants are selected and again mutated, and the second generation of mutants is again tested to identify new mutants where the property is further improved. Thus, traditional directed evolution methods randomly search through the sequence space of a polymer one residue at a time to identify mutants with an increased fitness.

Such traditional methods are limited, however, by the finite capacity of existing assays to screen mutants. Existing screening assays may observe and/or select from between about 10³ or 10¹² mutants, depending on the particular method. However, for a typical protein of 300 amino acid residues the number of possible amino acid combinations is about 10³⁹⁰. Thus, screening assays can only observe a small fraction of sequences in the sequence space of a given parent.

Using the analytical methods described in Section 5.2, therefore, a user can improve upon such existing methods by identifying those polymer residues having the highest level of structural tolerance and specifically selecting those residues for mutation in a directed evolution experiment. In preferred embodiments a user may select residues that have a structural tolerance (or a parameter such as site entropy which is indicative thereof) above a threshold value, which may also be selected by a user (for example, based on the number of residues that can be reasonably targeted in a particular experiment). For example, a user may select residues having a structural tolerance (or site entropy, etc.) that is above the average value of structural tolerance values in the parent sequences site entropy distribution. Alternatively, a user may select residues whose structural tolerance is greater than, e.g., one standard deviation in the site entropy distribution.

According to the invention, mutations of residues that have an increased structural tolerance value and/or fewer coupling interactions with other residues are less likely to destablize the structure of the polymer and, conversely, are more likely to increase the polymer's fitness. Accordingly, by focusing the mutations in a directed evolution experiment to residues having higher structure tolerance values, the number of sequences that must be tested or screened is considerably reduced and the sequence space may be searched more efficiently using existing screening techniques.

In preferred embodiments where the parent sequence is a protein or other polypeptide sequence, the parent sequence (and mutants thereof) may be expressed in facile gene expression systems to obtain libraries of mutant proteins. Any source of nucleic acid in purified form can be utilized as the starting nucleic acid. Thus, the process may employ DNA or RNA, including messenger RNA. The DNA or RNA may be either single or double stranded. In addition, DNA-RNA hybrids which contain one strand of each may be utilized. The nucleic acid sequence may also be of various lengths depending on the size of the sequence to be mutated. Preferably, the specific nucleic acid sequence is from 50 to 50,000 base pairs. It is contemplated that entire vectors containing the nucleic acid encoding the protein of interest may be used in these methods.

Any specific nucleic acid sequence can be used to produce a population of mutants by these processes. Most preferably, in the invention, mutants of a parent sequence are generated by saturation or site directed mutagenesis techniques. These methods are targeted to specifically mutate selected residues, which, e.g., have higher structural tolerance or site entropy values. Oligonucleotide-directed mutagenesis, which replaces a short sequence with a synthetically mutagenized oligonucleotide may also be employed to generate evolved polynucleotides having improved expression. An initial population of mutants of a specific (i.e., parent) sequence may be created by methods known in the art. These methods include oligonuclotide-directed mutagenesis, error-prone PCR, DNA shuffling, parallel PCR, chemical mutagenesis and sexual PCR.

Nucleic acid or DNA shuffling, which uses a method of in vitro or in vivo, generally homologous, recombination of pools of nucleic acid fragments or polynucleotides, can be employed to generate polynucleotide molecules having variant sequences of the invention.

Once the evolved polynucleotide molecules are generated they can be cloned into a suitable vector selected by the skilled artisan according to methods well known in the art. If a mixed population of the specific nucleic acid sequence is cloned into a vector it can be clonally amplified by inserting each vector into a host cell and allowing the host cell to amplify the vector. The mixed population may be tested to identify the desired recombinant nucleic acid fragment. The method of selection will depend on the DNA fragment desired. For example, in this invention a DNA fragment which encodes for a protein with improved properties can be determined by tests for functional activity and/or stability of the protein. Such tests are well known in the art.

Using the methods of directed evolution, the invention provides a novel means for producing functional proteins with improved properties. If desired, the mutants can be expressed in conventional or facile expression systems such as E. coli. Conventional tests can be used to determine whether a protein of interest produced from an expression system has improved expression, folding and/or functional properties. For example, to determine whether a polynucleotide subjected to directed evolution and expressed in a foreign host cell produces a protein with improved activity, one skilled in the art can perform experiments designed to test the functional activity of the protein. Briefly, the evolved protein can be rapidly screened, and is readily isolated and purified from the expression system or media if secreted. It can then be subjected to assays designed to test functional activity of the particular protein in native form. Such experiments for various proteins are well known in the art, and are discussed in the Examples below.

5.4. Implementation Systems and Methods

Computer System.

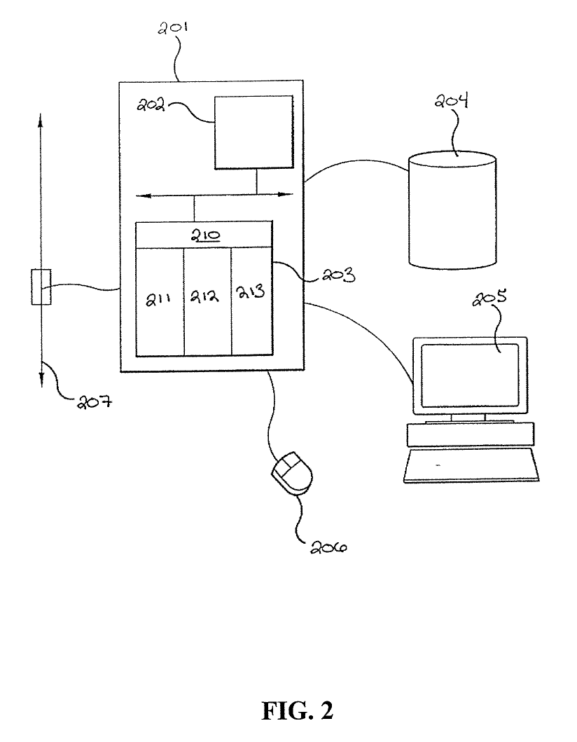

The analytical methods described in the previous subsections may preferably be implemented by the use of one or more computer systems, such as those described herein. Accordingly, FIG. 2 schematically illustrates an exemplary computer system suitable for implementation of the analytical methods of this invention. Computer 201 is illustrated here as comprising internal components linked to external components. However, a skilled artisan will readily appreciate that one or more of the components described herein as “internal” may, in alternative embodiments, be external. Likewise, one or more of the “external” components described here may also be internal. The internal components of this computer system include processor element 202 interconnected with a main memory 203. For example, in one preferred embodiment computer system 201 may be a Silicon Graphics R10000 Processor running at 195 MHz or greater and with 2 gigabytes or more of physical memory. In another, less preferable, exemplary embodiment, computer system 201 may be an Intel Pentium based processor of 150 MHz or greater clock rate and the 32 megabytes or more of main memory.

The external components may include a mass storage 204. This mass storage may be one or more hard disks which are typically packaged together with the processor and memory Such hard disks are typically of at least 1 gigabyte storage capacity, and more preferably have at least 5 gigabytes or at least 10 gigabtyes of storage capacity. The mass storage may also comprise, for example, a removable medium such as, a CD-ROM drive, a DVD drive, a floppy disk drive (including a Zip™ drive), or a DAT drive or other. Other external components include a user interface device 205, which can be, for example, a monitor and a keyboard. In preferred embodiments the user interface is also coupled with a pointing device 206 which may be, for example, a “mouse” or other graphical input device (not illustrated). Typically, computer system 201 is also linked to a network link 207, which can be part of an Ethernet or other link to one or more other, local computer systems (e.g., as part of a local area network or LAN), or the network link may be a link to a wide area communication network (WAN) such as the Internet. This network link allows computer system 201 to communicate with one or more other computer systems.

Typically, one or more software components are loaded into main memory 203 during operation of computer system 201. These software components may include both components that are standard in the art and special to the invention, and the components collectively cause the computer system to function according to the analytical methods of the invention. Typically, the software components are stored on mass storage 204 (e.g., on a hard drive or on removable storage media such as on one or more CD-ROMs, RW-CDs, DVDs, floppy disks or DATs). Software component 210 represents an operating system, which is responsible for managing computer system 201 and its network interconnections. This operating is typically an operating system routinely used in the art and may be, for example, a UNIX operating system or, less preferably, a member of the Microsoft Windows™ family of operating systems (for example, Windows 2000, Windows Me, Windows 98, Windows 95 or Windows NT) or a Macintosh operating system. Software component 211 represents common languages and functions conveniently present in the system to assist programs implementing the methods specific to the invention. Languages that may be used include, for example, FORTRAN, C, C++ and less preferably JAVA. The analytical methods of the invention may also be programmed in mathematical software packages which allow symbolic entry of equations and high-level specification of processing, including algorithms to be used, thereby freeing a user of the need to procedurally program individual equations and algorithms. Examples of such packages include Matlab from Mathworks (Natick, Massachusetts), Mathematica from Wolfram Research (Champaign, Ill.) and S-Plus from Math Soft (Seattle, Wash.). Accordingly, software component 212 represents the analytic methods of the invention as programmed in a procedural language or symbolic package. The memory 203 may, optionally, further comprise software components 213 which cause the processor to calculate or determine a three-dimensional structure for a macromolecule and, in particular, for a given polymer sequence such as a protein or nucleic acid sequence. Such programs are well known in the art, and numerous software packages are available. This software includes Swiss-Pdb Viewer (Glaxo Wellcome Experimental Research); Biograf (Molecular Simulations, Inc); O (generally used for crystallography); Explorer (MSI); Quenta, CHARMM; and Sybil (Tripos). The memory may also comprise one or more other software components, such as one or more other files representing, e.g., one or more sequences of polymer residues including, for example, a parent sequence and/or other sequences (for example, mutant sequences) in a sequence space. The memory 203 may also comprise one or more files representing the three-dimensional structures of one or more sequences, including a file representing the three-dimensional structure of a parent sequence, such as a parent protein or nucleic acid.

Computer Program Products

The invention also provides computer program products which can be used, e.g., to program or configure a computer system for implementation of analytical methods of the invention. A computer program product of the invention comprises a computer readable medium such as one or more compact disks (i.e., one or more “CDs”, which may be CD-ROMs or a RW-CDs), one or more DVDs, one or more floppy disks (including, for example, one or more ZIP™ disks) or one or more DATs to name a few. The computer readable medium has encoded thereon, in computer readable form, one or more of the software components 212 that, when loaded into memory 203 of a computer system 201, cause the computer system to implement analytic methods of the invention. The computer readable medium may also have other software components encoded thereon in computer readable form. Such other software components may include, for example, functional languages 211 or an operating system 210. The other software components may also include one or more files or databases including, for example, files or databases representing one or more polymer sequences (e.g. protein or nucleic acid sequences) and/or files or databases representing one or more three-dimensional structures for particular polymer sequences (e.g. three-dimensional structures for proteins and nucleic acids.

System Implementation

In an exemplary implementation, to practice the methods of the invention a parent sequence may first be loaded into the computer system 201. For example, the parent sequence may be directly entered by a user from monitor and keyboard 205 and by directly typing a sequence of code of symbols representing different residues (e.g., different amino acid or nucleotide residues). Alternatively, a user may specify parent sequences, e.g., by selecting a sequence from a menu of candidate sequences presented on the monitor or by entering an accession number for a sequence in a database (for example, the GenBank or SWISPROT database) and the computer system may access the selected parent sequence from the database, e.g., by accessing a database in memory 203 or by accessing the sequence from a database over the network connection, e.g., over the internet.

The programs may then cause the computer system to obtain a three-dimensional structure of the parent sequence. For example, the three-dimensional structure for the parent sequence may also be accessed from a file (for example, a database of structures) in the memory 203 or mass storage 204. Alternatively, the three-dimensional structure may also be retrieved through the computer network (e.g., over the network) from a database of structures such as the PDB database. In yet other embodiments, the software components may, themselves, calculate a three-dimensional structure using the molecular modeling software components. Such software components may calculate or determine a three-dimensional structure, e.g., ab initio or may use empirical or experimental data such as X-ray crystallography or NMR data that may also be entered by a user of loaded into the memory 203 (e.g., from one or more files on the mass storage 204 or over the computer network 207). The software components may further cause the computer system to calculate a conformational energy for the parent sequence using the three-dimensional structure.

Finally, the software components of the computer system, when loaded into memory 203, preferably also cause the computer system to determine a structural tolerance or, in the alternative, a parameter related to or correlating with structural tolerance according to the methods described herein. For example, the software components may cause the computer system to generate one or more mutant sequences of the parent and, using the conformation determined or obtained for the parent sequence, determine the conformational energy of each mutant and identifying mutants that are compatible with the parent sequence's conformational energy. The structural tolerance of the residues may then be determined, e.g., by determining the site entropy sᵢ. Alternatively, the software components may cause the computer system to determine or evaluate the solvent accessibility of each residue in the parent sequence using its three-dimensional conformation.

Upon implementing these analytic methods, the computer system preferably then outputs, e.g., the structural tolerance or structural tolerance values of residues of the parent sequence. For instance, the structural tolerance values and/or values of one or more other parameters relating to or correlating with structural tolerance (for example, the site entropy value and/or solvent accessibility values) may be output to the monitor, printed on a printer (not shown) and/or written on mass storage 204. In preferred embodiments, the software components may also cause the computer system to select and identify one or more particular residues in the parent sequence for mutation, e.g., in a directed evolution experiment. For example, the computer system may identify residues of the parent sequence having a structural tolerance value that is above a certain threshold, such as values above the average structural tolerance value for residues of the polymer or, alternatively, values above one or more standard deviations of the average structural tolerance value. These residues could be identified, for a user, as ones which, if mutated, are most likely to improve properties of the polymer in a directed evolution experiment.

Alternative systems and methods for implementing the analytic methods of this invention are also intended to be comprehended within the accompanying claims. In particular, the accompanying claims are intended to include the alternative program structures for implementing the methods of this invention that will be readily apparent to those skilled in the relevant art(s).

### 6. EXAMPLES

The invention is also described by means of particular examples. However, the use of such examples anywhere in the specification is illustrative only and in no way limits the scope and meaning of the invention or of any exemplified term. Likewise, the invention is not limited to any particular preferred embodiments described herein. Indeed, many modifications and variations of the invention will be apparent to those skilled in the art upon reading this specification and can be made without departing from its spirit and scope. The invention is therefore to be limited only by the terms of the appended claims along with the full scope of equivalents to which the claims are entitled.

6.1. Effects of Coupling in Directed Evolution

This example describes computational experiments which investigate how coupling between residues of a biopolymer (e.g., amino acid residues of a protein) influences an evolutionary search. A general description of a fitness landscape (F) that models the effect of coupling between pairs of residues has been previously described (see, Saven & Wolynes, J. Phys. Chem. B. 1997, 101:8375). Specifically, this fitness landscape comprises two-body terms, which model the effect of coupling between pairs of residues, added to an uncoupled fitness contribution for each residue.

$$F = {\sum }_{i = 1}^{N} f ( {i}_{a} ) + b {\sum }_{i = 1}^{N - 1} {\sum }_{j > i}^{N} f ( {i}_{a} . {j}_{a} ) {λ}_{ij} \qquad ( Equation 12 )$$

where N is the number of residues, iₐ is the identity of residue i (i.e., the amino acid at position i in a protein), f(iₐ) is the uncoupled fitness contribution of iₐ to the total fitness (F), and f(iₐ,jₐ) is the fitness contribution of coupling interactions between amino acid residues iₐ and jₐ. The constant b is the relative strength of the coupled and uncoupled interactions. If residues i and j are coupled, λᵢⱼ=1, otherwise λᵢⱼ=0. Similar fitness approximations that use one- and two-body terms are known in the art and have been used previously to model thermal stability (see, e.g., Dahiyat & Mayo, Science 1997, 278:82; Malakaukas & Mayo, Nature Structural Biology 1998, 5:470; Saven & Wolynes, J. Phys. Chem. B. 1997, 101, 8375; Abkevich et al., J. Mol. Biol. 1995, 252, 460; Li et al., Science 1996, 273, 666).

To investigate how coupling influences an evolutionary search, a directed evolution experiment was performed in silico for a hypothetical biopolymer having N=50 residues. Specifically, fitness contribution values f(iₐ) and f(iₐ, jₐ) were randomly assigned from a Gaussian distribution for each residue iₐ, and for each pair of residues {iₐ,jₐ}. Values of λ were assigned so that τ randomly selected pairs of residues had a value λᵢⱼ=1. All other residue pairs had a value λᵢⱼ=0. For example, when τ=50 the experiment simulates a directed evolution experiment using a parent biopolymer (e.g., a protein) having coupling interactions between 50 randomly selected pairs of residues.

The hypothetical biopolymer was “mutated” by selected a residue i at random and changing its amino acid identity. 3000 mutants were thus identified and “screened” by evaluating each mutant's fitness F according to Equation 12 above. The directed evolution algorithm gradually progressed through different “fitness heights” (i.e., different levels of fitness) on the fitness landscape.

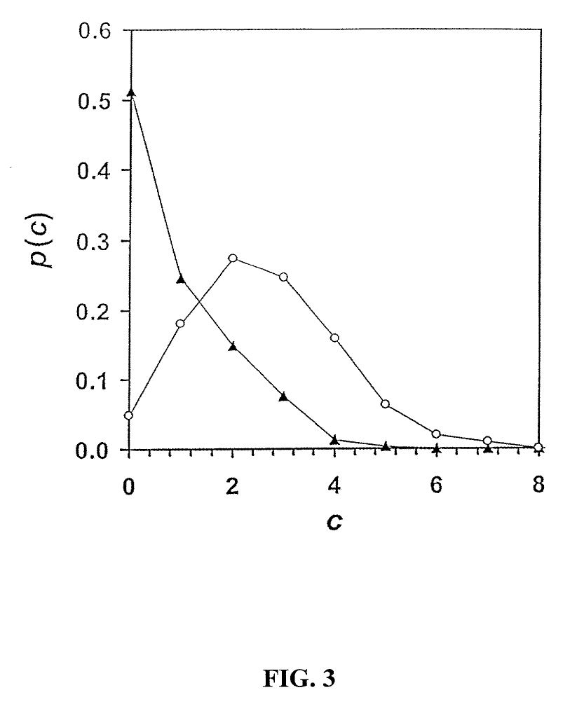

At each round of evolution, the coupling of the residues where beneficial mutations occurred was recorded. FIG. 3 provides a plot showing the probability P(c) that a positive mutation (i.e., a mutation that increases the protein's fitness, F) occurs at a residue having c coupled interactions. The probability distribution is shown for two different fitness values as the polypeptide progressively ascends the “fitness landscape” during screening: F=0.0 (∘); and F=17.0 (▴). The probability of a positive mutation occurring at a highly coupled residue decreases significantly as the overall fitness F of the polypeptide increases.

Without being limited to any particular theory or mechanism of interaction, these results are believed to be caused by the finite sampling size of the screening step during in vitro directed evolution. In particular, only a very limited number of mutations may be screened in a typical directed evolution experiment. However, when a mutation is made at a coupled residue, it is necessary to improve, not only the uncoupled term, f(iₐ), but also coupled terms f(iₐ, jₐ) of the fitness profile for every residues j that is coupled to residue i (i.e., for all j∈{λᵢⱼ=1}). The probability of improving both the coupled and uncoupled terms of the fitness profile decreases, however, as the sequence become more highly optimized (i.e., as F becomes higher). Thus, the probability of a beneficial mutation occurring at an uncoupled, rather than a coupled, residue increases as the sequence becomes more optimized.

It is noted that the results described here are independent of the specific form used to describe or model the fitness landscape F for a polymer (e.g., for a biopolymer such as DNA, RNA or a polypeptide). In particular, one skilled in the art can readily obtain the same results using any model that incorporates a variable degree of coupling between residues. Examples of other models which may be used include Kauffman's NK-model (Kauffman, The Origins of Order 1993, Oxford University Press, Oxford), a lattice protein model (Li et al., Science 1996, 273:666; Shakhnovich, Phys. Rev. Lett. 1994, 72:3907) and RNA secondary structure models (Fontana & Shuster, Science 1998, 280:1451). In addition, the fitness landscape is not limited to forms having only a two-body coupling term. Expressions may also be used which include terms for interactions or couplings between multiple (e.g., three or more) residues. Indeed, such terms will be useful and desirable in embodiments where a user wishes to also consider more complicated coupling interactions (for example, buried hydrophobic surface areas and/or complicated electrostatic interactions).

6.2. Calculating Structural Tolerance of Proteins

The computational experiments described in Section 6.1, supra, demonstrate that strategies of directed evolution which concentrate mutagenesis on residues having weak coupling interactions are most likely to show improvement. The present example extends this result to make experimentally relevant predictions. In particular, this example describes the use of a detailed protein design model that calculates interactions between amino acid residues for two actual proteins: subtilisin E (Jain et al., J. Mol. Biol. 1998, 284:137-144) and T4 lysozyme (Matsumura et al., J. Biol. Chem. 1989,264:16059). Using this model, a site entropy term is calculated for each residue position of the two proteins, and this term is used to identify particular residues in the two proteins which are most tolerant of mutations.

Materials and Methods

Force Field and Amino Acid Side Chain Rotamer Library.

Typically, conformational energy and fitness are independently evaluated according to the invention. To improve fitness, conformational energy is retained. The energy term used in this example consisted of two contributions: a rotamer/backbone contribution e(iᵣ), and a rotamer/rotamer contribution e(iᵣ, jₛ):

$$E = {\sum }_{i = 1}^{N} ⅇ ( {i}_{r} ) + {\sum }_{i = 1}^{N - 1} {\sum }_{j > 1}^{N} ⅇ ( {i}_{r} , {j}_{s} ) \qquad ( Equation 13 )$$

where N is the number of residues (e.g., amino acid residues in a protein) and iᵣ is rotamer r at position i. Because the backbone remains fixed in this model, its energy contribution is not relevant to evaluating fitness, and therefore was not included in Equation 13.

Potential functions and parameters for van der Walls interactions, hydrogen bonding, and electrostatics were used as previously described (see, Dahiyat & Mayo, Proc. Natl. Acad. Sci. U.S.A. 1997,94:10172; and Dahiyat & Mayo, Protein Science 1996,5:895). For atomic radii and internal coordinate parameters, the DREIDING force field was used (Mayo et al., J. Phys. Chem. 1990, 94:8897). The van der Waals energies were modeled using a standard 6-12 Leonard-Jones potential with an additional 0.9 scale factor applied to the atomic radii to soften the lack of flexibility implied by the fixed backbone and rotamer descriptions. A ceiling of 500 kcal/mol was set for the rotamer/rotamer energies to avoid unhindered van der Waals contributions and to expedite mean-field convergence. All rotamer/backbone and rotamer/rotamer energies were computed using 10 Silicon Graphics R 10000 processors running at 195 MHz, and stored prior to the mean-field calculation.

Subtilisin and Lysozyme.

A backbone-dependent rotamer library was used to model protein structures, as described by Dunbrack and Karplus (Dunbrack & Karplus, J. Mol. Biol. 1993, 230:543; Dunbrack & Karplus, Nature Structural Biology 1994,1:334). However, modifications were made as previously described (Dahiyat et al, Protein Science 1997, 6:1333). Specifically, the χ₃ angles that were undetermined from database statistics were assigned the following values: Arg, −60°, 60° and 180°; Gln, −120°, −60°, 0°, 60°, 120° and 180°; Glu, 0°, 60° and 120°; Lys, −60°, 60° and 180°. Rotamers having combinations of χ₃ and χ₄ that resulted in sequential g⁺/g⁻ or g⁻/g⁺ angles were eliminated from the calculation.

Rotamers interacting with the backbone with energies greater than either 5 kcal/mol (subtilisin E) or 20 kcal/mol (T4 lysozyme) were eliminated from the calculation. Amino acid residues 1-4 and 269-274 of subtilisin E were fixed in the wild-type amino acid side chain conformations. For subtilisin E, an average of 121 rotamers per residue were considered, corresponding to about 3.2×10⁴ one-body energies, 5.1×10⁸ two-body energies and a rotamer space of 10⁴⁹⁷ combinations. For T4 lysozyme, an average of 176 rotamers per residue were considered, corresponding to 2.9×10⁴ one-body energies, 4.1×10⁸ two-body energies, and a rotamer space of 10³⁸⁴ combinations.

Antibody 4-4-20.

In another example, a rotamer model was used to study antibody 4-4-20, a known anti-fluorescyl antibody. Rotamers that interact with the backbone at energies greater than 5 kcal/mol were eliminated from the calculation. For antibody 4-4-20, the entropy of each residue of the light (V_{L}) and heavy (V_{H}) chains was determined (228 residues total). An average of 140 rotamers per residue were considered, corresponding to 3.3×10⁴ one-body energies, 5.3×10⁸ two-body energies, and a rotamer space of 10⁴³⁹ combinations. The high-resolution crystal structure was obtained. See, Whitlow, M., Ioward, A. J., Wood, J. F., Voss, E. W., Hardman, K. D. (1995). 1.85-angstrom structure of antifluorescein 4-4-20-FAB, Protein Engineering, 8: 749-761.

The mean-field minimization was run until a final temperature of T=700 K was reached. This solution required 10000 minutes on a single Silicon Graphics R10000 processor running at 195 MHZ and 2.1 gigabytes of physical memory.

A directed evolution-type experiment was run to improve the binding of antibody 4-4-20 to fluorescein. See, Boder, E. T., Midelfort, K. S., Wittrup, K. D. (2000). Directed evolution of antibody fragments with monovalent femtomolar antigen-binding affinity, Proc. Natl. Acad. Sci. USA, 97: 10701-10705. In this experiment, yeast-displayed mutant libraries of antibodies were created and run over a column. The antibodies that bound tightly to the fluorescein were harder to wash from the column. Four rounds of increasing stringency (the level of required binding was increased) were performed and sets of improved mutants were isolated. Some mutations occurred only a few times in each data set and are considered neutral. Others occurred in less stringent rounds and became fixed as the stringency was increased. These mutations were considered essential for improved binding.

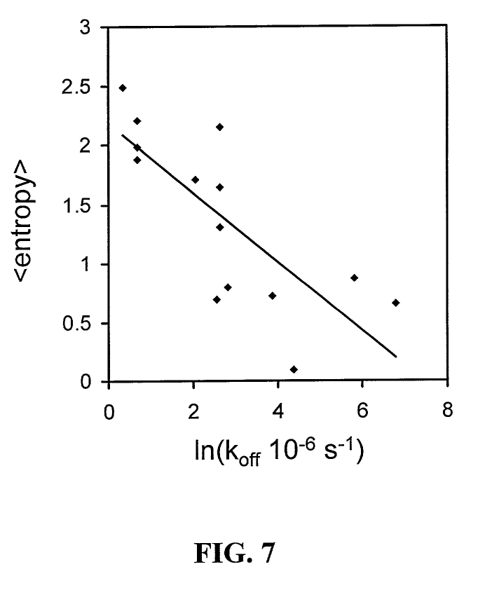

The average entropy was plotted for mutations discovered in each round of improved stringency (excluding the neutral mutations). As the fitness of the parent sequence increases, mutations are more concentrated at the high-entropy residues of the antibody. In addition, the standard deviation in entropies decreases as the fitness increases. Together, these results indicate that, when the parent sequence is highly optimized, the beneficial mutations can be reliably found at the high-entropy positions. Further, these results represent an experimental verification of the dynamics of directed evolution discovered using the generic statistical model (FIG. 7).

As the parent sequence becomes more optimized, the probability that a beneficial mutation will occur at a highly coupled residue decreases dramatically. Using this analysis, the evolutionary potential of an experiment can also be assessed. By calculating the entropy of residues at which mutations are made, the number of generations before the optimum is reach can be estimated. As shown in FIG. 7, the off-rate k_{off} of the 4-4-20 antibody mutants is plotted versus the entropy of the sites where the beneficial mutations occurred. The term is a kinetic constant for the antibody, which is an expression if its affinity for the fluorescein antigen. As the antibody is mutated, the value for k_{off} may change, as shown in FIG. 7. The best mutants bind with femtomolar affinities (the left of the graph). As the fitness of the parent sequence increases, there is a trend towards mutations occurring at high entropy residues.

Mean-field Theory.

Searching an entire sequence space for a global fitness optimum remains computationally and experimentally intractable. To circumvent these difficulties, statistical mechanics methods were used to evaluate the coupling at each residue position of the two proteins, using structural tolerance of a position towards amino acid substitutions as an indirect measure of the coupling.

“Structural tolerance” is preferably quantitated by counting the number of sequences (also referred to as “states”) Ω that are compatible with a conformational energy. In preferred embodiments, the structural tolerance is measured by the “sequence entropy”, S=k_{B}·lnΩ (see, Saven & Wolynes, J. Phys. Chem. B 1997, 101:8375). The “site entropy” sᵢ is a measure of the variability of the amino acid residue identity at position i among the different sequences consistent with a given energy E. Preferably, the site entropy is obtained or determined from the probability p(iₐ) that an amino acid residue having the identity iₐ exists at site i of the polypeptide; e.g., by the formula.

$${s}_{i} = - {k}_{B} {\sum }_{a = 1}^{A} p ( {i}_{a} ) \cdot ln p ( {i}_{a} ) \qquad ( Equation 14 )$$

where A is the total number of amino acids, and k_{B} is preferably chosen to be unity (i.e., k_{B}=1). The amino acid probabilities p(iₐ) may be calculated as the sum of the amino acid residue's rotamer probabilities, as determined by the mean-field theory methods described in Section 6.2.1, supra., or by keeping residues at other sites in their wild-type amino acid identities

The mean field solution of Equation 13, supra, is

$$\begin{matrix}E = {\sum }_{i = 1}^{N} {\sum }_{r = 1}^{{K}_{1}} {e}_{mf} ( {i}_{r} ) p ( {i}_{r} ) where & ( Equation 15 ) \\ {e}_{mf} ( {i}_{r} ) = ⅇ ( {i}_{r} ) + {\sum }_{j \neq i}^{N} {\sum }_{s = 1}^{{K}_{j}} ⅇ ( {i}_{r} , {j}_{s} ) p ( {j}_{s} ) . & ( Equation 16 )\end{matrix}$$

The term e_{mf}(iᵣ) is the mean-field energy felt by rotamer r at position i, and Kᵢ and Kⱼ are the total number of rotamers at residues i and j, respectively (see, Saven et al., J. Phys. Chem B 1997, 101:8375; Lee, J. Mol. Biol. 1994, 236:918; Koehl & Delarue, J. Mol. Biol. 1994, 239:249; Koehl & Delarue, Current Opinion in Structural Biology 1996, 6:222; Lee and Subbiah, J. Mol. Biol. 1991, 217, 373). The term p(jₛ) is the probability that rotamer s exist at residue j, and is calculated at a “temperature” T, iterating between:

$$p ( {j}_{s} ) = \frac{{ⅇ}^{- β {e}_{mf} ( {j}_{s} )}}{{\sum }_{{s}^{′} = 1}^{{K}_{j}} {ⅇ}^{- β {e}_{mf} ( {j}_{{s}^{′}} )}} \qquad ( Equation 17 )$$

where β=1/k_{B}T, and k_{B} is Boltzmann's constant. Typically for this work, k_{B}=1.

To calculate mean-field energies, the probability vectors p(jₛ) were initially set to 1/Kⱼ, and the mean field energies were calculated from Equation 16 for each residue. These mean-field energies where then used to recalculated p(jₛ) according to Equation 17, and the algorithm iterated between Equations 16 and 17 until self-consistency is achieved. Convergence was significantly improved if the probability vector p is updated with a memory of the previous step as described by Lee (J. Mol. Biol. 1994, 236:918).

An initially high temperature value (T=50,000 K) was set, and the convergence algorithm was repeated as the temperature was lowered in increments of 100 K to a final temperature value of T=600 K (for subtilisin E) or T=300 K (for T4 lysozyme) was reached. These final temperatures are not physical temperatures; in this mean field model they correspond to an estimated energy above which the structural stability of the proteins is compromised.

The mean-field solution for subtilisin E required 8900 minutes on a single Silicon Graphics R10000 Processor running at 195 MHz and 2.1 gigabytes of physical memory. The mean-field solution for T4 lysozyme required 6402 minutes on the same computer system.

Mean-Field Derivations.

Entropy in a mean-field model of the invention can be calculated from the probability distribution of allowed amino acid substitutions. The entropy sᵢ for a given site i is calculation from Equation 14, also discussed above:

$${s}_{i} = - {k}_{B} {\sum }_{a = 1}^{A} p ( {i}_{a} ) \cdot ln p ( {i}_{a} ) \qquad ( Equation 14 )$$

where A is the total number of amino acids, p(iₐ) is the probability that amino acid a exists at position i, and k_{B} is taken to be 1. If all amino acids are equally likely, then sᵢ=ln A≈3.0. The total sequence entropy is simply the sum of the site entropies,

$$S ( F ) = {\sum }_{i}^{N} {s}_{i} . \qquad ( Equation 18 )$$

The mean-field theory is applied to calculate the amino acid probabilities required by Equation 14, as a function of the fitness. It is difficult, however, to do this with a fixed fitness. Instead, the thermodynamic equivalence of groups or ensembles can be used to work with a fixed fitness <F>_{A}, where the average is taken over all sequences corresponding to a “temperature” T. The “temperature” acts to generate a “variational” free energy. This means that the energy of the system can be changed or mathematically controlled via the “temperature” variable, as shown for example by these derivations.

Thus, the variational free energy:

G=(F)_{A-TS} (Equation 20)

is minimized subject to the normalization condition for all i:

$${\sum }_{a}^{A} p ( {i}_{a} ) = 1. \qquad ( Equation 21 )$$

The average energy is obtained from:

$${⟨ F ⟩}_{A} = {\sum }_{i}^{N} {⟨ f ( {i}_{a} ) ⟩}_{A} + \frac{b}{2} {\sum }_{i}^{N} {\sum }_{j \neq 1}^{N} {⟨ f ( {i}_{a} , {i}_{b} ) ⟩}_{A} {λ}_{ij} \qquad ( Equation 22 )$$

where the averages are taken over all amino acids at each position. Utilizing the mean-field approximation, this can be rewritten as:

$${⟨ F ⟩}_{A} = {\sum }_{i}^{N} {\sum }_{a}^{A} p ( {i}_{a} ) f ( {i}_{a} ) + \frac{b}{2} {\sum }_{i}^{N} {\sum }_{j \neq i}^{N} {\sum }_{a}^{A} {\sum }_{b}^{A} p ( {i}_{a} ) p ( {j}_{a} ) f ( {i}_{a} , {j}_{b} ) {λ}_{ij} . \qquad ( Equation 23 )$$

Introducing the Lagrange multiplier μᵢ in the normalization of the probabilities for each site, the variational free energy is:

$$G ( T ) = {\sum }_{i}^{N} {\sum }_{a}^{A} p ( {i}_{a} ) f ( {i}_{a} ) + \frac{b}{2} {\sum }_{i}^{N} {\sum }_{j \neq 1}^{N} {\sum }_{a}^{A} {\sum }_{b}^{A} p ( {i}_{a} ) p ( {j}_{b} ) f ( {i}_{a} , {j}_{b} ) {λ}_{ij} + {k}_{B} T {\sum }_{i}^{N} {\sum }_{a}^{A} p ( {i}_{a} ) ln p ( {i}_{a} ) + {\sum }_{i}^{N} {μ}_{i} ( {\sum }_{a}^{A} p ( {i}_{a} ) - 1 ) . \qquad ( Equation 24 )$$

Minimization of G is performed by setting the partial derivative ∂E/∂p(iₐ) to zero for all i and a. After rearrangement, this gives:

p(iₐ)=exp[−β≢(iₐ)−1−βμᵢ] (Equation 25)

where β=1/k_{B}T and

$$ϵ ( {i}_{a} ) ≡ γ ( {i}_{a} ) + \frac{b}{2} {\sum }_{j \neq i}^{N} {\sum }_{b}^{A} p ( {j}_{b} ) f ( {i}_{a} , {j}_{b} ) {λ}_{ij} . \qquad ( Equation 26 )$$

By solving for μᵢ using the normalization condition (Equation 21), the following partition function is obtained:

$$\begin{matrix}{ⅇ}^{βμ + 1} = {\sum }_{a}^{A} {ⅇ}^{- βϵ ( {i}_{a} )} ≡ {Z}_{i} \\ and therefore , & ( Equation 27 ) \\ p ( {i}_{a} ) = \frac{{ⅇ}^{- βϵ ( {i}_{a} )}}{{Z}_{i}} . & ( Equation 28 )\end{matrix}$$

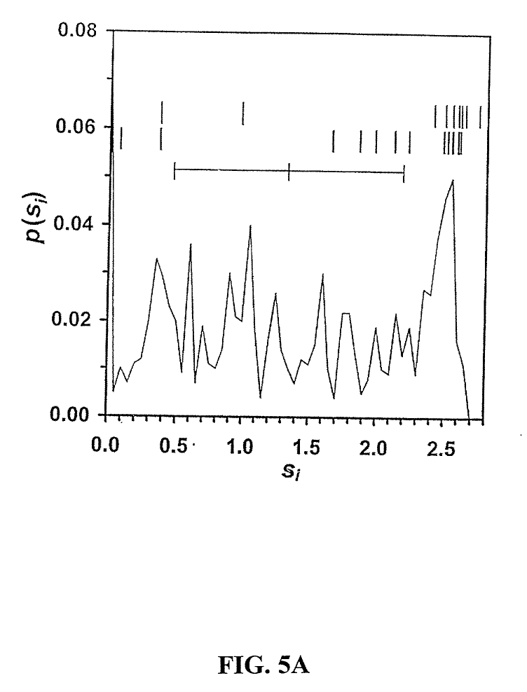

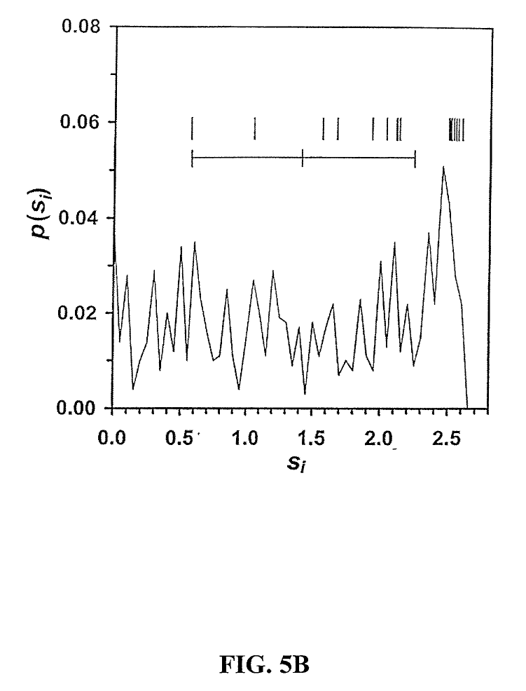

Equations 26 and 28 constitute a set of self-consistent equations for p(iₐ). Self-consistency is computed by iterating between these equations until the probabilities converge, according to a convergence criterion. When solving the self-consistent equations, decreasing the temperature is analogous to increasing the fitness. As the fitness is increased, the number of sequences consistent with that fitness decreases, thus decreasing the total entropy. The probabilities calculated as the “temperature” decreases are used to calculate the average fitness and entropy See, Equations 20 and 24. See also, FIG. 5. The list of sequences consistent with a “fitness” demonstrates the tolerance of each position to amino acid substitutions, as measured by the site entropies.

Results

The protein backbones of subtilisin E (274 amino acid residues) and T4 lysozyme (164 amino acid residues) were retrieved from high-resolution crystal structures (Matsummura et al., J. Biol. Chem. 1989, 264:16059; Jain et al., J. Mol. Biol. 1998, 284:137-144), and interactions between residues were calculated by coarse graining the flexibility of each amino acid residue into rotamers, and constructing a force field to calculate the rotamer/backbone and rotamer stabilizing energies (see, Section 6.2.1, supra). By initially eliminating certain rotamers from the calculation (as described in Section 6.2.1), it became computationally tractable to evaluate Equation 26, supra, for a given amino acid sequence. However, the total sequence space which must be searched is still hyper-astronomically large: 10³⁴³ amino acid sequence combinations for subtilisin E, and 10²¹⁴ amino acid sequence combinations for T4 lysozyme.

The site entropy sᵢ is calculated for all possible amino acid residue substitutions at position i, as well as for the amino acid residue at position i of the parent. Site entropy values were calculated for both subtilisin E and T4 lysozyme according to Equation 28. A tabulation of the site entropy at each amino acid residue position in subtilisin E is shown in FIG. 4A. A corresponding plot for percent solvent exposed is shown in FIG. 4B. The distributions of site entropy values P(s,) are provided in FIGS. 5A-B for both subtilisin E (FIG. 5A) and for T4 lysozyme (FIG. 5B). In this preferred embodiment, the site entropy s, is a measurement or indication of the number of amino acid residue substitutions that can be made at each residue i without disrupting the protein's structure. Specifically, those residue positions that are intolerant of mutations will have a low site entropy, whereas a residue position that is tolerant for mutations will have a relatively high value for it site entropy.

6.3. Correlation of Structural Tolerance with Directed Evolution

This example tests the predictions made in the Example presented in Section 6.2, supra, and verifies that beneficial mutations to a biopolymer are preferably made by directed evolution at structurally tolerant residue positions. In particular, the site entropy calculations for subtilisin E and T4 lysozyme were compared to mutations found from previous evolution experiments on those proteins (see, in particular, Zhao & Arnold, Protein Engineering 1999, 12:47; Chen & Arnold, Proc. Natl. Acad. Sci. USA. 1993, 90:5618; You & Arnold, Protein Engineering 1996, 9:77-83; and Pjura et al., Protein Science 1993, 2:2217). The comparisons are illustrated in FIGS. 5A-B.

In particular, FIG. 5A provides a plot of the distribution profile P(sᵢ) of site entropy values calculated for subtilisin E (see Section 6.2, supra). The top row of horizontal bars indicates mutations found from the in vitro evolution of subtilisin E in a screen for improved thermostability while retaining protein activity (Zhao & Arnold, Protein Engineering 1999, 12:47) These amino acid residue positions are listed in Table 1, below, along with their calculated site entropy values sᵢ. Similarly, FIG. 5B provides a plot of the distribution profile of site entropy values calculated for T4 lysozyme. The row of horizontal bars on this plot indicates in vitro mutations to the enzyme that improved stability (Pjura et al., Protein Science 1993, 2:2217). The amino acid positions are listed in Table 2, below, along with their calculated site entropy values sᵢ.

Seven out of the nine mutations that improved subtilisin E thermal stability occur at positions computed to be highly tolerant (i.e., positions having high site entropy values). The stabilizing mutations discovered by the evolution of T4 lysozyme also preferentially occur at positions of high site entropy. Thus, the entropy predictions would aid in an evolutionary search to improve thermostability of both these proteins, demonstrating that the computational methods described here are valid independent of the specific protein or experimental screening protocol used.

In some embodiments of directed evolution, it may be desirable to improve a property other than thermostability of a biopolymer. Nevertheless, in embodiments where the desired property is at least correlated with stability, the thermostability E may be used as an indicator of fitness F, and structure-based entropy predictions as described in Section 6.2, supra, may be used. Thus, for example, proteins having enhanced activity at high temperatures may be identified by improving thermostability (see, e g., Zhao & Arnold, Protein Engineering 1999, 12:47; Giver et al., Proc. Natl. Acad. Sci. U.S.A. 1998, 95:12809-12813). Indeed, in directed evolution experiments where libraries of subtilisin E mutants were screened for both improved thermal stability and enhanced activity, mutations were identified that improved both properties. Similarly, activity and stability are highly correlated in screens for T4 lysozyme. See, Zhao & Arnold, Protein Engineering 1999, 12: 47; Giver et al, Proc. Natl. Acad. Sci. USA 1998,95: 12809-12813; Pejura et al, Protein Science 1993,2: 2217 Activity improving mutations therefore may also be found at positions which are tolerant for mutations.

Similarly, mutations that improve other properties are also be biased towards “high entropy” positions of a polypeptide. As an example, Table 3 below lists amino acid residue positions of subtilisin E where mutations improved that enzyme's reactivity in the organic solvent dimethyl formamide (Chen & Arnold, Proc. Natl. Acad. Sci. U.S.A. 1993, 90:5618; You & Arnold, Protein Engineering 1996, 9:77-83), along with the site entropy value sᵢ calculated for each residue. These mutations and their site entropy values are also indicated by the lower row of vertical bars in the site entropy distribution profile P(sᵢ) for subtilisin E shown in FIG. 5A. As with thermal stability, the mutations are strongly biased towards amino acid residues that have low site entropy values. Indeed, mutations at amino acid residues 181 and 218 produce mutants that have both enhanced thermal stability and enhanced activity in organic solvent.

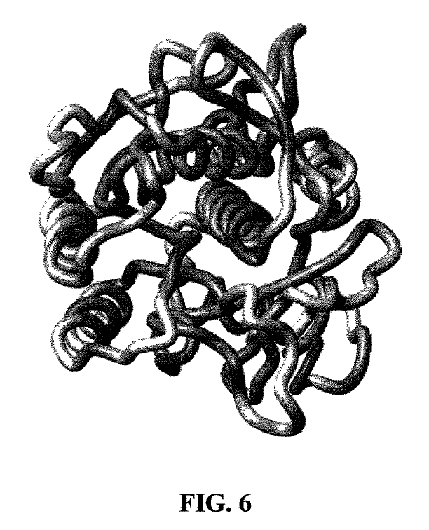

FIG. 6 illustrates the three dimensional structure of subtilisin E, with the site entropy profile of each amino acid residue indicated by its color. In particular, the yellow amino acid residues have the highest site entropy values (2.16<sᵢ<3.00; i.e., greater than about one standard deviation above the mean) and are therefore the most variable. The red residues have intermediate site entropy values (1.31<sᵢ<2.16; i.e., between the mean value and about one standard deviation above) and are moderately variant. The gray residues are ones having below average entropy values (sᵢ<1.31). FIG. 6 also illustrates a general trend towards sites having the highest entropy profile begin located on the protein's surface and exposed to solvent. By contrast, amino acid residues that are more conserved and have lower site entropy values are generally located in the core of the protein and are shielded from solvent. Thus the level of a residue's exposure or accessibility to solvent may also be used to identify those residues that are likely to be more tolerant to mutations in a directed evolution experiment. However, solvent accessibility is a secondary measure or indication of physical features that may lead to a residue's tolerance to mutations, whereas the site entropy is calculated directly from fundamental features that produce such tolerance. Typically, the positions with high site entropy values (e.g., greater than about one standard deviation above the mean site entropy value for residues of a particular biopolymer) and below average solvent accessibility (e.g., less than about 24%) are close to the protein surface and have their side chains only partially burried in the protein core. Thus, both the site entropy and solvent accessibility may be used to identify residues for mutation in directed evolution experiments. However, solvent accessibility is a less preferable parameter than site entropy. Using the mean-filed computations described in Section 6.2, supra, energies and site entropy values may be calculated based on all amino acid substitutions, and not merely on the wild-type amino acid identity as in solvent accessibility calculations. Thus, solvent accessibility is a useful but less preferred measurement for identifying residues that may be tolerant to mutations, e.g., in a directed evolution experiment.

To further illustrate this point, Table 4, below, compares site entropy values and solvent accessibilities at positions in the subtilisin E and T4 lysozyme proteins where positive mutations are found. Solvent accessibility was determined as described above, e.g. according to Lee and Richards (1971) J. Mol. Biol. 55, 379. Although most positive mutations are found at sites exposed to the solvent, some positive mutations are located at site with poor solvent accessibility. The site entropy does indicate, however, that these sites will be tolerant to mutations. As an example, amino acid residue 107 in subtilisin E has an above average site entropy value (sᵢ=1.62), but very poor solvent accessibility (˜1%). This residue, which is an isoleucine in the wild-type protein, is located on an α-helix with its side chain oriented towards and completely burried within the protein core. However, the packing of the side chains of the surrounding residues is such that several other amino acid residues may be sustained without affecting conformational energy. Mean-field theory calculations, described in Section 6.2, supra, indicate that acceptable amino acid residues at this position (and their probabilities, p) include: isoleucine (p=0.42), cysteine (p=0.23), valine (p=0.12), methionine (p=0.09), aspartic acid (p=0.03), threonine (p=0.01), serine (p=0.01) and alanine (p=0.01). In directed evolution experiments, the mutation of Ile₁₀₇→Val increased subtilisin E activity in organic solvent.

Similarly, amino acid residue 151 in T4 lysozyme (a threonine in the wild-type protein) is located on an α-helix near the protein surface, and is partially blocked from the solvent by surrounding atoms. Consequently, this amino acid residue has below average solvent accessibility (˜17%). However, its site entropy value is above average (sᵢ=1.53). Mean-field theory calculations also predict several other amino acid residues that are compatible at this positions, including: methionine (p=0.37), leucine (p=0.34), cysteine (p=0.11), glutamic acid (p=0.09), glutamine (p=0.05), aspartic acid (p=0.03), serine (p=0.01), and threonine (p=0.01). In directed evolution experiments, the mutation of Thr₁₅₁→Ser increased the protein's activity.

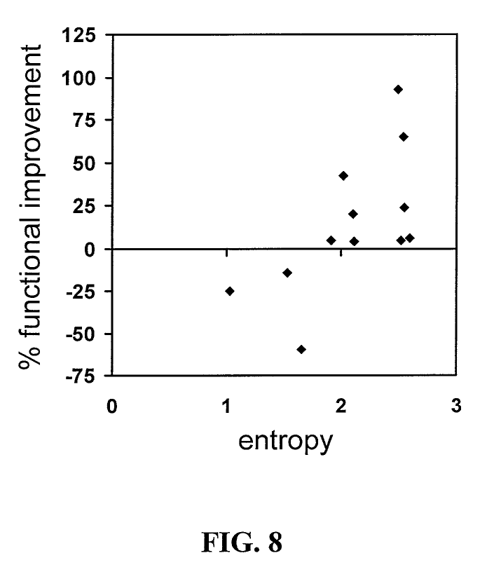

The T4 lysozyme calculations can also be represented in graphic form, as shown for example in FIG. 8. The percent functional improvement is plotted versus the entropy, as determined from the T4 lysozyme calculation. The functional improvement is measured as halo formation on a bacterial lawn and is compared against the wild-type halo formation (0%). For this system, the largest improvements in catalytic activity occurred at the high-entropy positions. Thus, targeting the high entropy positions increases the probability of finding the largest gains in catalytic activity.

TABLE 1
Mutated Residues That Improve
Subtilisin E Thermal Stability

| Amino Acid Residue | Sᵢ |
|---|---|
| 9 | 2.55 |
| 14 | 2.50 |
| 76 | 2.45 |
| 118 | 2.37 |
| 161 | 2.69 |
| 166 | 0.96 |
| 181 | 0.36 |
| 194 | 2.59 |
| 218 | 2.54 |

TABLE 2
Mutated Residues That Improve
T4 Lysozyme Thermal Stability

| Amino Acid Residue | Sᵢ |
|---|---|
| 14 | 2.59 |
| 16 | 2.02 |
| 22 | 1.66 |
| 26 | 1.03 |
| 40 | 2.54 |
| 41 | 1.91 |
| 93 | 2.52 |
| 113 | 2.54 |
| 116 | 2.50 |
| 119 | 2.11 |
| 147 | 2.10 |
| 151 | 1.53 |
| 153 | 0.55 |
| 163 | 2.49 |

TABLE 3
Mutated Residues That Improve Subtilisin E Activity in
Dimethyl Formamide

| Amino Acid Residue | Sᵢ |
|---|---|
| 48 | 2.09 |
| 60 | 0.0 |
| 97 | 0.06 |
| 103 | 2.48 |
| 107 | 1.62 |
| 131 | 2.43 |
| 156 | 2.19 |
| 181 | 0.36 |
| 182 | 1.81 |
| 188 | 2.50 |
| 206 | 1.94 |
| 218 | 2.54 |
| 255 | 2.54 |

TABLE 4A
Comparison of Site Entropies and Solvent Accesibility
of Subtilisin E Amino Acid Residues

| Amino Acid Residue | Site Entropy (sᵢ) | %-Solvent Exposure |
|---|---|---|
| 9 | 2.55 | 56 |
| 14 | 2.50 | 34 |
| 48 | 2.09 | 20 |
| 60 | 0.00 | 0 |
| 76 | 2.45 | 46 |
| 97 | 0.06 | 19 |
| 103 | 2.48 | 61 |
| 107 | 1.62 | 1 |
| 118 | 2.37 | 79 |
| 131 | 2.43 | 37 |
| 156 | 2.19 | 53 |
| 161 | 2.69 | 92 |
| 166 | 0.96 | 8 |
| 181 | 0.36 | 23 |
| 182 | 1.81 | 52 |
| 188 | 2.50 | 88 |
| 194 | 2.59 | 71 |
| 206 | 1.94 | 40 |
| 218 | 2.54 | 50 |
| 255 | 2.54 | 41 |

TABLE 4B
Comparison of Site Entropies and Solvent Accesibility
of T4 Lysozyme Amino Acid Residues

| Amino Acid Residue | Site Entropy (sᵢ) | %-Solvent Exposure |
|---|---|---|
| 14 | 2.59 | 47 |
| 16 | 2.02 | 53 |
| 22 | 1.66 | 19 |
| 26 | 1.03 | 2 |
| 40 | 1.03 | 2 |
| 41 | 1.91 | 34 |
| 93 | 2.52 | 81 |
| 113 | 2.54 | 69 |
| 116 | 2.50 | 51 |
| 119 | 2.11 | 54 |
| 147 | 2.10 | 50 |
| 151 | 1.53 | 17 |
| 153 | 0.55 | 0 |
| 163 | 2.49 | 63 |

6.4. Dead-End Elimination Methodology

Single Residues

Mean-field theory is an approximate method and is generally expected to worsen as the coupling in the system increases. See, Fischer, K. H., and Hertz, J. A., Spin Glasses, Cambridge University Press (1991). To overcome this problem, an algorithm can be used to calculate the entropy based on a series of minimizations performed by dead-end elimination. This dead-end elimination or “DEE” entropy algorithm calculates the substitution energy of all amino acids at all positions in the wild-type amino acid background. First, a residue is chosen (residue i) and the remaining residues in the structure are held in their wild-type amino acid identity. Then, residue i is assigned an amino acid identity a. The flexibility of all the amino acid side chains are resolved into rotamers, and the global minimum energy conformation is found using the DEE algorithm. This minimum energy is then assigned to that amino acid at that residue. This procedure is used to find the energy of all twenty amino acid substitutions at residue i. The process is repeated for all residues in the protein, so the outcome of the algorithm is the energy for all single-mutant amino acid substitutions at every position.

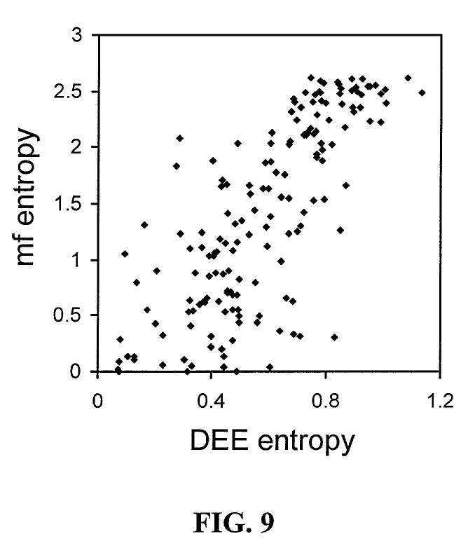

The probability of each amino acid at each residue is calculated from the energies by taking the Boltzmann weight of the energies at each position:

$$p ( {i}_{a} ) = \frac{{ⅇ}^{- β E ( {i}_{a} )}}{{\sum }_{b}^{A} {ⅇ}^{- β E ( {i}_{b} )}} \qquad ( Equation 29 )$$

where A=20 is the total number of amino acids, p(iₐ) is the probability of amino acid a existing at residue i, and E(iₐ) is the energy of amino acid a at residue i. The entropy sᵢ is then calculated by:

$${s}_{i} = - {k}_{B} {\sum }_{a}^{A} {p}_{i} ( a ) ln {p}_{i} ( a ) \qquad ( Equation 30 )$$

where k_{B} is Boltzmann's constant (here, k_{B}=1). When the entropies calculated by the mean-field algorithm and DEE-algorithm are compared, both algorithms agree on the assignment of the high entropy positions (FIG. 9). FIG. 9 shows a comparison of the entropy calculated by the mean-field algorithm and the DEE algorithm for T4 lysozyme. Both algorithms identify find the same high-entropy positions, but differ in their rank ordering of low-entropy positions. This is a demonstration of the fact that the mean-field approximation is less accurate at highly coupled positions. Disagreement increases for the low entropy residues. This is, in part, due to the mean-field assumption. As the coupling between residues increases, this assumption becomes less valid. Also, the restriction that the DEE algorithm must calculate the substitution energies based on the wild-type amino acid background leads to disagreement between the methods.

Multiple Residues

In single-residue saturation experiments, targeting residues that have a high entropy implies that the beneficial mutations found at these positions will be additive. This search strategy was constructed based on the discovery that the probability of improving non-additive interactions is negligible when the wild-type sequence is highly optimized and the number of mutants that can be screened is small. However, in high-throughput multiple-site saturation experiments, the high mutagenesis rate and large mutant library increases the probability that a set of multiple mutations will be found that collectively contribute to a non-additive fitness improvement. An advantage of the DEE-entropy method is that the substitutability of multiple sites can be evaluated simultaneously. Instead of making 20 substitutions at a single residue i, m residues i₁, . . . , iₘ can be picked and 20^{m} mutations can be tested for each m-group of residues.

The computational challenge is to identify clusters of residues that are collectively coupled, but remain uncoupled from the remainder of the residues in the enzyme. The invention employs an algorithm that exhaustively determines the optimal set of m residues to be targeted using multi-site combinatorial mutagenesis. First, all of the clusters of m-coupled residues are determined. Two residues are considered to be coupled if their side-chains are coupled (they have at least one rotamer that interacts above a threshold of 5 kcal/mol). Second, at each m-cluster of residues all combinations of amino acids are substituted, and the minimum-energy conformation of each combination is determined using the Dead-End Elimination algorithm. During the energy calculation, the remaining residues in the enzyme are held in their wild-type amino acid identity, but the conformation of the side-chains are allowed to adjust to the mutations at the clustered residues. For each of the m-residue clusters, the algorithm generates a list of 20^{m} energies corresponding to all of the possible amino acid substitutions. This gives the advantage of being able to study several approaches to targeting the optimal residues. For example, it is possible to target the cluster that has the highest entropy, the most amino acid substitutions that lead to energies more stable than wild-type, or the lowest energy amino acid combination.

What is claimed is:

1. A method for selecting residues of a particular polymer sequence for mutation, comprising the steps of:
(a) obtaining a level of structural tolerance for residues of the particular polymer sequence; and
(b) selecting structurally tolerant residues for mutation.

2. The method of claim 1 wherein the particular polymer sequence comprises a sequence of amino acid residues.

3. The method of claim 1 wherein the particular polymer sequence comprises a sequence of nucleotide residues.

4. The method of claim 1 wherein the selected structurally tolerant residues are residues having a level structural tolerance above a threshold level.

5. The method of claim 1 wherein the selected structurally tolerant residues are residues having a level of structural tolerance greater than an average level of structural tolerance for residues of the particular polymer sequence.

6. The method of claim 5 wherein the selected structurally tolerant residues are residues having a level of structural tolerance at least one standard deviation above the average level.

7. The method of claim 1 wherein the level of structural tolerance is calculated for one or more residues of the particular polymer sequence.

8. The method of claim 1 wherein the level of structural tolerance of a particular residue in the particular polymer sequence is related to the number of polymer sequences, in a sequence space containing the particular polymer sequence, that:
(i) have a mutation at the particular residue; and
(ii) are compatible with a conformational energy of the particular polymer sequence.

9. The method of claim 1 wherein the level of structural tolerance of a particular residue in the particular polymer sequence is related to the number of other polymer sequences that:
(i) are identical to the particular polymer sequence except that the identity of the particular residue in each other polymer sequence is different from the identity of said particular residue in the particular polymer sequence; and
(ii) are compatible with the conformational energy of the particular polymer sequence.

10. The method of claim 1 wherein the level of structural tolerance of a particular residue in the polymer sequence is related to the number or level of coupling interactions said particular residue has with other residues in the particular polymer sequence.

11. The method of claim 10 wherein the level of coupling interactions the particular residue has with other residues in the polymer sequence is provided by the contribution of said coupling interactions to a conformational energy for the particular polymer sequence.

12. The method of claim 1 wherein the level of structural tolerance of a particular residue to the particular polymer sequence is provided by a site entropy for the particular polymer sequence.

13. The method of claim 12 wherein the site entropy of the particular residues is related to the number of polymer sequences in a sequence space containing the particular polymer sequence, that:
(i) have a mutation at the particular residue; and
(ii) are compatible with a conformational energy E of the particular polymer sequence.

14. The method of claim 13 wherein the site entropy of the particular residue is obtained by a method which comprises:
(a) identifying compatible polymer sequences from a plurality of different polymer sequences, which compatible polymer sequences are compatible with the conformational energy E of the particular polymer sequence; and
(b) determining the number of said compatible sequences that have a mutation at the particular residues.

15. The method of claim 14 wherein a stochastic algorithm is used to identify compatible polymer sequences.

16. The method of claim 13 wherein the site entropy sᵢ the particular residue is obtained by a method which comprises determining the number of homologous polymer sequences that have a mutation at the particular residue.

17. The method of claim 13 where each polymer sequence that is compatible with the conformational energy E of the particular polymer sequence has, when folded into a backbone conformation corresponding to a backbone conformation of the particular polymer sequence, a conformational energy less than or approximately equal to the conformational energy E of the particular polymer sequence.

18. The method of claim 12 wherein the site entropy of the particular residue is related to the number of other polymer sequences that:
(i) are identical to the particular polymer sequence except that the identity of said particular residue in each other polymer sequence is different from the identity of said particular residue in the particular polymer sequence; and
(ii) are compatible with the conformational energy E of the particular polymer sequence.

19. The method of claim 18 where each polymer sequence that is compatible with the conformational energy E of the particular polymer sequence h as, when folded into a backbone conformation corresponding to a backbone conformation of the particular polymer sequence, a conformational energy less than or approximately equal to the conformational energy E of the particular polymer sequence.

20. The method of claim 1 wherein the level of structural tolerance for a particular residue in the particular polymer sequence is provided by the solvent accessibility of said particular residue.

21. A method for selecting residues at particular sites of a polymer sequence for mutation, comprising the steps of:
(a) obtaining a conformational energy for the particular polymer sequence;
(b) obtaining conformational energies for a plurality of other polymer sequences, which other polymer sequences comprise the particular polymer sequence with at least one mutation;
(c) identifying compatible polymer sequences from the plurality of other polymer sequences, which compatible polymer sequences have a conformational energy consistent with the conformational energy for the particular polymer sequence;
(d) obtaining a structural tolerance for one or more particular residues in the particular polymer sequence, wherein the structural tolerance of a particular residue is related to the number of compatible polymer sequences in which the particular residue is mutated,
wherein a particular residue is selected for mutation if said particular residue has a high level of structural tolerance.

22. The method of claim 21 wherein the conformational energy for the particular polymer sequence is determined from a three-dimensional structure of said particular polymer sequence.

23. The method of claim 21 wherein conformational energies for the plurality of other polymer sequences are determined from a three-dimensional structure for the particular polymer sequence.

24. The method of claim 21 wherein the other polymer sequences are identical to the particular polymer sequence except that the identity of the particular residue in each other polymer sequence is different from the identity of said particular residue in the particular polymer sequence.

25. A computer system for analyzing a polymer sequence, comprising:
a memory; and
a processor interconnected with the memory and having one or more software components loaded therein,
wherein the one or more software components cause the processor to execute steps of a method according to claim 1.

26. The computer system of claim 25 wherein the software components comprise a database of polymer sequences.

27. The computer system of claim 25 wherein the software components comprise a database of three-dimensional structures for polymer sequences.

28. A computer program product comprising a computer readable medium having one or more software components encoded thereon in computer readable form, wherein the one or more software components may be loaded into a memory of a computer system and cause a processor interconnected with said memory to execute steps of a method according to claim 1.

29. A computer program product according to claim 28 wherein the computer readable medium further has, encoded thereon in computer readable form, a database of polymer sequences.

30. A computer program product according to claim 28 wherein the computer readable medium further has, encoded thereon in computer readable form, a database of three-dimensional structures for polymer sequences.

31. A method for directed evolution of a polymer, comprising the steps of:
(a) providing a parent polymer sequence, which parent polymer sequence has one or more properties of interest;
(b) selecting one or more structurally tolerant residues of the parent polymer sequence for mutation.
(c) generating, from the parent polymer sequence, one or more mutant polymer sequences in which the one or more selected residues are mutated; and
(d) screening the one or more mutant sequences for the one or more properties of interest.

32. A method according to claim 31 which method is iteratively repeated, and wherein at least one mutant sequence selected in a first iteration is the parent sequence in a second iteration.

33. The method of claim 31 wherein a property of interest is catalytic activity.

34. The method of claim 31 wherein a property of interest in binding to a particular ligand or substrate.

35. The method of claim 31 wherein a property of interest in thermal stability.

36. The method of claim 31 wherein a property of interest is binding specificity.

37. The method of claim 31 wherein a property of interest is enantio-specificity.

38. The method of claim 31 wherein the parent polymer sequence is a sequence of amino acid residues.

39. The method of claim 31 wherein the parent polymer sequence is a sequence of nucleotide residues.

40. A method for directed evolution of a polymer, comprising the steps of:
(a) providing a parent polymer sequence, which parent polymer sequence has one or more properties of interest;
(b) selecting one or more residues of the parent polymer sequence for mutation, which one or more residues are selected according to claim 1.
(c) generating, from the parent polymer sequence, one or more mutant polymer sequences in which the one or more selected residues are mutated;
(d) screening the one or more mutant sequences for the one or more properties of interest; and
(e) selecting at least one mutant sequence where one or more properties of interest are modified.

41. A method for directed evolution of a polymer, comprising the steps of:
(a) providing a parent polymer sequence, which parent polymer sequence has one or more properties of interest;
(b) selecting one or more residues of the parent polymer sequence for mutation, which one or more residues are selected according to claim 21;
(c) generating, from the parent polymer sequence, one or more mutant polymer sequences in which the one or more selected residues are mutated;
(d) screening the one or more mutant sequences for the one or more properties of interest; and
(e) selecting at least one mutant sequence where one or more properties of interest are modified.

42. The method of claim 21, wherein structural tolerance is obtained according to a determination of a site entropy for at least one site of the polymer.

43. The method of claim 42, wherein site entropy is determined for a plurality of sites.

44. The method of claim 43, wherein a plurality of sites are treated as a group, and a site entropy for the group is determined combinatorially.

45. A method of claim 31, further comprising the step of selecting at least one mutant sequence having a property of interest.

46. A method of claim 45, wherein the property of interest is modified.

47. A method of claim 45, wherein the property of interest is a property not shown by a parent sequence.

48. A method of claim 45, wherein the property is a catalytic activity.

49. A method of claim 47, wherein the property is a catalytic activity.

50. A method of claim 12, wherein the site entropy is determined by making a polymer residue mutation at a selected residue location and minimizing the side chains of one or more parent polymers at all other residue locations according to a minimization algorithm.

51. A method of claim 40, wherein the minimization algorithm comprises a mean-field algorithm.

52. A method of claim 40, wherein the minimization algorithm comprises a dead-end elimination algorithm.

53. A method of claim 50, wherein the polymer comprises a sequence of amino acid residues.

54. A method of claim 50, wherein a plurality of residue locations are mutated combinatorially.

55. A method of claim 53, wherein a plurality of residue locations are mutated combinatorially.

56. A method of claim 55, wherein a selected number of residue locations is identified as a cluster of residues, the states corresponding to a set of amino acid mutations at the residue locations comprising a cluster are treated together, and a site entropy for the mutated cluster is calculated.

57. A method of claim 55, wherein a selected number of residue locations m is identified as a cluster of residues, the 20^{m} states corresponding to all amino acid mutations at the residue locations comprising a cluster are treated together, and a site entropy for the mutated cluster is calculated.

58. A method of claim 56 wherein the site entropies of two or more clusters are compared.

59. A method of claim 57 wherein the site entropies of two or more clusters are compared.

60. A method of claim 12, wherein a selected number of residue locations is identified as a cluster of residues, the states corresponding to all residue mutations at the selected residue locations comprising a cluster are treated together, and a site entropy for the mutated cluster is calculated.
